SEQUENCE LISTING (41 sequences)

SEQ ID NO: 1 - protein, 9 aa, synthetic construct
  REGION 1..9: note = epitope
VVGAVGVGK

SEQ ID NO: 2 - protein, 6 aa, synthetic construct
  REGION 1..6: note = alpha chain CDR1 of TCR 3-14
NSAFQY

SEQ ID NO: 3 - protein, 6 aa, synthetic construct
  REGION 1..6: note = alpha chain CDR2 of TCR 3-14
TYSSGN

SEQ ID NO: 4 - protein, 14 aa, synthetic construct
  REGION 1..14: note = alpha chain CDR3 of TCR 3-14
CAMSAGQAGTALIF

SEQ ID NO: 5 - protein, 5 aa, synthetic construct
  REGION 1..5: note = beta chain CDR1 of TCR 3-14
SGHNS

SEQ ID NO: 6 - protein, 6 aa, synthetic construct
  REGION 1..6: note = beta chain CDR2 of TCR 3-14
FNNNVP

SEQ ID NO: 7 - protein, 13 aa, synthetic construct
  REGION 1..13: note = beta chain CDR3 of TCR 3-14
CASSPWTPDTQYF

SEQ ID NO: 8 - protein, 5 aa, synthetic construct
  REGION 1..5: note = alpha chain CDR1 of TCR 4-13
TSINN

SEQ ID NO: 9 - protein, 7 aa, synthetic construct
  REGION 1..7: note = alpha chain CDR2 of TCR 4-13
IRSNERE

SEQ ID NO: 10 - protein, 14 aa, synthetic construct
  REGION 1..14: note = alpha chain CDR3 of TCR 4-13
CATDRNSGNTPLVF

SEQ ID NO: 11 - protein, 5 aa, synthetic construct
  REGION 1..5: note = beta chain CDR1 of TCR 4-13
SGHAT

SEQ ID NO: 12 - protein, 6 aa, synthetic construct
  REGION 1..6: note = beta chain CDR2 of TCR 4-13
FQNNGV

SEQ ID NO: 13 - protein, 13 aa, synthetic construct
  REGION 1..13: note = beta chain CDR3 of TCR 4-13
CASSLDSFYEQYF

SEQ ID NO: 14 - protein, 6 aa, synthetic construct
  REGION 1..6: note = alpha chain CDR1 of TCR 3-13
NSASDY

SEQ ID NO: 15 - protein, 7 aa, synthetic construct
  REGION 1..7: note = alpha chain CDR2 of TCR 3-13
IRSNMDK

SEQ ID NO: 16 - protein, 13 aa, synthetic construct
  REGION 1..13: note = alpha chain CDR3 of TCR 3-13
CAENNFGNEKLTF

SEQ ID NO: 17 - protein, 6 aa, synthetic construct
  REGION 1..6: note = beta chain CDR1 of TCR 3-13
DFQATT

SEQ ID NO: 18 - protein, 7 aa, synthetic construct
  REGION 1..7: note = beta chain CDR2 of TCR 3-13
SNEGSKA

SEQ ID NO: 19 - protein, 15 aa, synthetic construct
  REGION 1..15: note = beta chain CDR3 of TCR 3-13
CSASTSGRWYNEQFF

SEQ ID NO: 20 - protein, 6 aa, synthetic construct
  REGION 1..6: note = alpha chain CDR1 of TCR 3-7
NSAFQY

SEQ ID NO: 21 - protein, 6 aa, synthetic construct
  REGION 1..6: note = alpha chain CDR2 of TCR 3-7
TYSSGN

SEQ ID NO: 22 - protein, 14 aa, synthetic construct
  REGION 1..14: note = alpha chain CDR3 of TCR 3-7
CAMSVGTSGSRLTF

SEQ ID NO: 23 - protein, 7 aa, synthetic construct
  REGION 1..7: note = alpha chain CDR2 of TCR 3-10
IRSNVGE

SEQ ID NO: 24 - protein, 6 aa, synthetic construct
  REGION 1..6: note = beta chain CDR2 of TCR 3-7
FNNNVP

SEQ ID NO: 25 - protein, 13 aa, synthetic construct
  REGION 1..13: note = beta chain CDR3 of TCR 3-7
CASSSWSPDTQYF

SEQ ID NO: 26 - protein, 6 aa, synthetic construct
  REGION 1..6: note = alpha chain CDR1 of TCR 3-10
DSASNY

SEQ ID NO: 27 - protein, 7 aa, synthetic construct
  REGION 1..7: note = alpha chain CDR2 of TCR 3-10
IRSNVGE

SEQ ID NO: 28 - protein, 16 aa, synthetic construct
  REGION 1..16: note = alpha chain CDR3 of TCR 3-10
CAATPISNFGNEKLTF

SEQ ID NO: 29 - protein, 5 aa, synthetic construct
  REGION 1..5: note = beta chain CDR1 of TCR 3-10
MNHEY

SEQ ID NO: 30 - protein, 6 aa, synthetic construct
  REGION 1..6: note = beta chain CDR2 of TCR 3-10
SMNVEV

SEQ ID NO: 31 - protein, 13 aa, synthetic construct
  REGION 1..13: note = beta chain CDR3 of TCR 3-10
CASSLSIVNEQFF

SEQ ID NO: 32 - protein, 273 aa, synthetic construct
  REGION 1..273: note = alpha chain of TCR 4-13
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDRNSGNTPLVFGKGTRLSVIANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 33 - protein, 311 aa, synthetic construct
  REGION 1..311: note = beta chain of TCR 4-13
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLDSFYEQYFGPGTRLTVTEDLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRG

SEQ ID NO: 34 - protein, 273 aa, synthetic construct
  REGION 1..273: note = alpha chain of TCR 3-13
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAENNFGNEKLTFGTGTRLTIIPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 35 - protein, 310 aa, synthetic construct
  REGION 1..310: note = beta chain of TCR 3-13
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSASTSGRWYNEQFFGPGTRLTVLEDLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRG

SEQ ID NO: 36 - protein, 275 aa, synthetic construct
  REGION 1..275: note = alpha chain of TCR 3-14
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSAGQAGTALIFGKGTTLSVSSNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 37 - protein, 311 aa, synthetic construct
  REGION 1..311: note = beta chain of TCR 3-14
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSPWTPDTQYFGPGTRLTVLEDLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRG

SEQ ID NO: 38 - protein, 275 aa, synthetic construct
  REGION 1..275: note = alpha chain of TCR 3-7
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSVGTSGSRLTFGEGTQLTVNPDIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 39 - protein, 311 aa, synthetic construct
  REGION 1..311: note = beta chain of TCR 3-7
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSSWSPDTQYFGPGTRLTVLEDLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRG

SEQ ID NO: 40 - protein, 275 aa, synthetic construct
  REGION 1..275: note = alpha chain of TCR 3-10
MTSIRAVFIFLWLQLDLVNGENVEQHPSTLSVQEGDSAVIKCTYSDSASNYFPWYKQELGKGPQLIIDIRSNVGEKKDQRIAVTLNKTAKHFSLHITETQPEDSAVYFCAATPISNFGNEKLTFGTGTRLTIIPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 41 - protein, 310 aa, synthetic construct
  REGION 1..310: note = beta chain of TCR 3-10
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLSIVNEQFFGPGTRLTVLEDLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRG

(19) United States
(12) Patent Application Publication
(10) Pub. No.: US 2025/0101078 A1
(43) Pub. Date: Mar. 27, 2025

(54) ANTIGEN RECOGNIZING CONSTRUCT THAT BINDS SPECIFIC PEPTIDE WITH DETERMINABLE AFFINITY AND T CELL RECEPTOR HAVING ANTIGENIC SPECIFICITY FOR KRAS AS WELL AS CORRESPONDING NUCLEIC ACID SEQUENCE, VECTOR, HOST CELL, PHARMACEUTICAL COMPOSITION AND KIT

(71) Applicants: T-Knife GmbH, Berlin (DE); Max-Delbrueck-Centrum für Molekulare Medizin in der Helmholtz-Gemeinschaft, Berlin (DE)

(72) Inventors: Lucia PONCETTE, Berlin (DE); Elisa KIEBACK, Berlin (DE); Jennifer ODURO, Berlin (DE); Arunraj DHAMODARAN, Berlin (DE); Lorenz KNACKSTEDT, Berlin (DE); Alexei LELIAVSKI, Berlin (DE); Thomas BLANKENSTEIN, Berlin (DE)

(21) Appl. No.: 18/730,453

(22) PCT Filed: Jan. 23, 2023
(86) PCT No.: PCT/EP2023/051554

§ 371 Date: Jul. 19, 2024

(30) Foreign Application Priority Data
Jan. 21, 2022 (EP) 22152794.8

(51) Int. Cl. C07K 14/725; A61K 38/00

(52) U.S. Cl. CPC C07K 14/7051; A61K 38/00

(57) ABSTRACT

The present invention inter alia relates to an antigen recognizing construct that binds to a specific peptide with determinable affinity, a T cell receptor having antigenic specificity for KRAS, a nucleic acid sequence encoding said antigen recognizing construct or T cell receptor, a vector comprising said nucleic acid sequence, and a host cell comprising said antigen recognizing construct or T cell receptor. The invention also relates to the antigen recognizing construct or T cell receptor, the nucleic acid sequence, the vector or the host cell for use in medicine or for use in the prevention and/or treatment of a disease. Additionally, the present invention relates to a method of treating a disease as well as a pharmaceutical composition and a kit.

### CROSS-REFERENCE TO RELATED APPLICATIONS

The present application claims the benefit of priority of European Patent Application No. 22152794.8, filed Jan. 21, 2022, the content of which is hereby incorporated by reference it its entirety for all purposes.

### FIELD OF THE INVENTION

The present invention relates to an antigen recognizing construct that binds the peptide VVGAVGVGK (SEQ ID NO: 1) with determinable affinity, a T cell receptor (TCR) having antigenic specificity for mutated KRAS, a nucleic acid sequence encoding said antigen recognizing construct or said T cell receptor, a respective vector comprising the nucleic acid sequence and a host cell comprising the antigen recognizing construct or the T cell receptor. The invention also relates to the antigen recognizing construct or the T cell receptor, the nucleic acid sequence, the vector, or the host cell, for use in medicine or for use in the prevention and/or treatment of a disease. The present invention further relates to a use of the antigen recognizing construct or the T cell receptor, the nucleic acid sequence, the vector, or the host cell, for the manufacture of a medicament for treating a disease. The present invention also relates to a method of treating a disease, comprising the step of administering a therapeutically effective amount of the antigen recognizing construct or the T cell receptor, the nucleic acid sequence, the vector, or the host cell. Additionally, the present invention also relates to a pharmaceutical composition comprising the antigen recognizing construct or the T cell receptor, the nucleic acid sequence, the vector, or the host cell of the invention and also to a respective kit for use in medicine.

### BACKGROUND OF THE INVENTION

Ras family proteins are small GTPases that are involved with transmitting signals within cells, including, for example, transduction of cell proliferation. Exemplary RAS proteins include KRAS (also called C-K-RAS, CFC2, K-RAS2A, KRAS2B, K-RAS4A, K-RAS4B, KI-RAS, KRAS1, KRAS2, NS, NS3, RALD, RASK2, K-ras, KRAS proto-oncogene, GTPase, and c-Ki-ras2), HRAS, and NRAS. Mutations in RAS proteins that disrupt negative growth signaling can lead to continuous proliferation of the cell.

When activated, mutated KRAS binds to guanosine-S-triphosphate (GTP) and converts GTP to guanosine 5′-diphosphate (GDP). The mutated KRAS protein product may be constitutively activated.

Mutated KRAS protein may be expressed in any of a variety of human cancers such as, for example, pancreatic cancers (e.g., pancreatic carcinoma), colorectal cancers, lung cancers (e.g., lung adenocarcinoma), endometrial cancers, ovarian cancers (e.g., epithelial ovarian cancer), prostate cancers, melanomas, thyroid cancers, and breast cancers, as well as some instances of myeloid leukemias such as AML.

There is thus a need for novel therapies targeting mutant RAS proteins. Accordingly, it is an object of the invention to provide such therapies.

### SUMMARY OF THE INVENTION

This object is inter alia accomplished by the antigen recognizing constructs, the TCRs, nucleic acids, vectors, host cells, methods, compositions, and kits having the features of the respective independent claims.

In a first aspect, the invention provides an antigen recognizing construct that binds the peptide VVGAVGVGK (SEQ ID NO: 1) with determinable affinity, wherein the antigen recognizing construct comprises a complementary determining region 3 (CDR3) of the α-chain having at least 90%, at least 91%, at least 92%, at least 93%, at least 94%, at least 95%, at least 96%, at least 97%, at least 98% or at least 99% sequence identity to an amino acid sequence selected from the group consisting of SEQ ID NOs: 10, 16, 4, 22, and 28, and/or wherein the antigen recognizing construct comprises a complementary determining region 3 (CDR3) of the β-chain having at least 90%, at least 91%, at least 92%, at least 93%, at least 94%, at least 95%, at least 96%, at least 97%, at least 98% or at least 99% sequence identity to an amino acid sequence selected from the group consisting of SEQ ID NOs: 13, 19, 7, 25, and 31.

In a second aspect, the invention provides a T cell receptor (TCR) having antigenic specificity for mutated KRAS, wherein the TCR comprises:
(a) an α-chain CDR1 comprising or consisting of the amino acid sequence of SEQ ID NO: 8,
an α-chain CDR2 comprising or consisting of the amino acid sequence of SEQ ID NO: 9,
an α-chain CDR3 comprising or consisting of the amino acid sequence of SEQ ID NO: 10;
a β-chain CDR1 comprising or consisting of the amino acid sequence of SEQ ID NO: 11,
a β-chain CDR2 comprising or consisting of the amino acid sequence of SEQ ID NO: 12, and
a β-chain CDR3 comprising or consisting of the amino acid sequence of SEQ ID NO: 13; or
(b) an α-chain CDR1 comprising or consisting of the amino acid sequence of SEQ ID NO: 14,
an α-chain CDR2 comprising or consisting of the amino acid sequence of SEQ ID NO: 15,
an α-chain CDR3 comprising or consisting of the amino acid sequence of SEQ ID NO: 16;
a β-chain CDR1 comprising or consisting of the amino acid sequence of SEQ ID NO: 17,
a β-chain CDR2 comprising or consisting of the amino acid sequence of SEQ ID NO: 18, and
a β-chain CDR3 comprising or consisting of the amino acid sequence of SEQ ID NO: 19; or
(c) an α-chain CDR1 comprising or consisting of the amino acid sequence of SEQ ID NO: 2,
an α-chain CDR2 comprising or consisting of the amino acid sequence of SEQ ID NO: 3,
an α-chain CDR3 comprising or consisting of the amino acid sequence of SEQ ID NO: 4,
a β-chain CDR1 comprising or consisting of the amino acid sequence of SEQ ID NO: 5,
a β-chain CDR2 comprising or consisting of the amino acid sequence of SEQ ID NO: 6, and
a β-chain CDR3 comprising or consisting of the amino acid sequence of SEQ ID NO: 7; or
(d) an α-chain CDR1 comprising or consisting of the amino acid sequence of SEQ ID NO: 20,
an α-chain CDR2 comprising or consisting of the amino acid sequence of SEQ ID NO: 21,
an α-chain CDR3 comprising or consisting of the amino acid sequence of SEQ ID NO: 22;
a β-chain CDR1 comprising or consisting of the amino acid sequence of SEQ ID NO: 23,
a β-chain CDR2 comprising or consisting of the amino acid sequence of SEQ ID NO: 24, and
a β-chain CDR3 comprising or consisting of the amino acid sequence of SEQ ID NO: 25; or
(e) an α-chain CDR1 comprising or consisting of the amino acid sequence of SEQ ID NO: 26,
an α-chain CDR2 comprising or consisting of the amino acid sequence of SEQ ID NO: 27,
an α-chain CDR3 comprising or consisting of the amino acid sequence of SEQ ID NO: 28;
a β-chain CDR1 comprising or consisting of the amino acid sequence of SEQ ID NO: 29,
a β-chain CDR2 comprising or consisting of the amino acid sequence of SEQ ID NO: 30, and
a β-chain CDR3 comprising or consisting of the amino acid sequence of SEQ ID NO: 31.

In a third aspect, the invention provides a nucleic acid sequence encoding the antigen recognizing construct or the T cell receptor.

In a fourth aspect, the invention provides a vector comprising the nucleic acid sequence.

In a fifth aspect, the invention provides a host cell comprising the antigen recognizing construct or the T cell receptor, the nucleic acid sequence, or the vector of the invention.

In a sixth aspect, the invention provides the antigen recognizing construct or the T cell receptor, the nucleic acid sequence, the vector, or the host cell, of the invention for use in medicine or for use in the prevention and/or treatment of a disease.

In a seventh aspect, the invention provides a use of the antigen recognizing construct or the T cell receptor, the nucleic acid sequence, the vector, or the host cell, for the manufacture of a medicament for treating a disease.

In an eighth aspect, the invention provides a method of treating a disease, comprising the step of administering a therapeutically effective amount of the antigen recognizing construct or the T cell receptor, or the nucleic acid sequence, or the vector, or the host cell of the invention.

In a ninth aspect, the present invention provides a pharmaceutical composition comprising the antigen recognizing construct or the T cell receptor, the nucleic acid sequence, the vector, or the host cell of the invention.

In a tenth aspect, the present invention provides a kit for use in medicine comprising the antigen recognizing construct or the T cell receptor, or the nucleic acid sequence, or the vector, or the host cell of the invention.

### BRIEF DESCRIPTION OF THE DRAWINGS

The invention will be better understood with reference to the detailed description when considered in conjunction with the non-limiting examples and the drawings, in which:

FIG. 1 shows TCR- or mock-transduced peripheral blood leukocytes (PBLs) from a human donor, which were co-cultured with KRAS^{G12V} expressing cell lines (intact or transduced with HLA-A*11:01) or alone (“T cells”). PMA and ionomycin stimulation (“P/I”) was used as a positive control of T cell activation. IFNγ secretion in the culture supernatants upon T cell activation was measured by ELISA.

FIG. 2 shows TCR-transduced PBLs from a human donor, which were co-cultured with HLA-A*11:01 expressing cells loaded with the recognized peptide (VVGAVGVGK) at descending concentrations (10⁻⁶ M to 10⁻¹² M). IFNγ secretion in the culture supernatants upon T cell activation was measured by ELISA. Normalized IFNγ secretion curves are shown.

FIG. 3 shows expression of TCRs on the surface of TCR-transduced CD8+ T cells from two donors as analyzed by flow cytometry. TCR expression was measured by staining the cells with anti-mouse TCR β-chain or with the HLA-A*11:01-VVGAVGVGK dextramer (“Dex”). The TCR expression is represented as mean fluorescence intensity (MFI) normalized to CT1 TCR.

FIG. 4 shows TCR-transduced human PBLs, which were co-cultured with HLA-A*11:01-expressing cell lines: CFPAC-1 cells that express KRAS^{G12V} (“CFPAC-1-A11”) or K-562 cells loaded with the recognized peptide (VVGAVGVGK; 10⁻⁶ M) (“K562-A11+G12V”). Non-loaded K-562 cells served as a negative control (“K562-A11”). Protein secretion in the culture supernatants upon T cell activation was measured by a multi-analyte bead flow assay.

FIG. 5 shows SEQ ID NOs: 32 to 41, which depict the α- and β-chain full-length sequences of the TCRs 4-13, 3-13, 3-14, 3-7, and 3-10 of the invention. Variable regions are underlined, constant regions are in bold. The term “variable region” may also be called “variable domain” as used herein, both terms may be used synonymously. The term “constant region” may also be called “constant domain” as used herein, both terms may be used synonymously.

### DETAILED DESCRIPTION OF THE INVENTION

As explained above, in a first aspect, the invention is directed to an antigen recognizing construct that binds the peptide VVGAVGVGK (SEQ ID NO: 1) with determinable affinity, wherein the antigen recognizing construct comprises a complementary determining region 3 (CDR3) of the α-chain having at least 90%, at least 91%, at least 92%, at least 93%, at least 94%, at least 95%, at least 96%, at least 97%, at least 98%, or at least 99% sequence identity to an amino acid sequence selected from the group consisting of SEQ ID NOs: 10, 16, 4, 22, and 28, and/or wherein the antigen recognizing construct comprises a complementary determining region 3 (CDR3) of the β-chain having at least 90%, at least 91%, at least 92%, at least 93%, at least 94%, at least 95%, at least 96%, at least 97%, at least 98%, or at least 99% sequence identity to an amino acid sequence selected from the group consisting of SEQ ID NOs: 13, 19, 7, 25, and 31. Thus, the first aspect of the invention also comprises an antigen recognizing construct that binds the peptide VVGAVGVGK (SEQ ID NO: 1) with determinable affinity, wherein the antigen recognizing construct comprises a complementary determining region 3 (CDR3) of the α-chain having at least 90%, at least 91%, at least 92%, at least 93%, at least 94%, at least 95%, at least 96%, at least 97%, at least 98%, or at least 99% sequence identity to an amino acid sequence selected from the group consisting of SEQ ID NOs: 10, 16, 4, 22, and 28, or wherein the antigen recognizing construct comprises a complementary determining region 3 (CDR3) of the β-chain having at least 90%, at least 91%, at least 92%, at least 93%, at least 94%, at least 95%, at least 96%, at least 97%, at least 98%, or at least 99% sequence identity to an amino acid sequence selected from the group consisting of SEQ ID NOs: 13, 19, 7, 25, and 31. The first aspect of the invention may also be directed to an antigen recognizing construct that binds the peptide VVGAVGVGK (SEQ ID NO: 1) with determinable affinity, wherein the antigen recognizing construct comprises a complementary determining region 3 (CDR3) of the α-chain having at least 90%, at least 91%, at least 92%, at least 93%, at least 94%, at least 95%, at least 96%, at least 97%, at least 98%, or at least 99% sequence identity to an amino acid sequence selected from the group consisting of SEQ ID NOs: 10, 16, 4, 22, and 28, and wherein the antigen recognizing construct comprises a complementary determining region 3 (CDR3) of the β-chain having at least 90%, at least 91%, at least 92%, at least 93%, at least 94%, at least 95%, at least 96%, at least 97%, at least 98%, or at least 99% sequence identity to an amino acid sequence selected from the group consisting of SEQ ID NOs: 13, 19, 7, 25, and 31. The first aspect of the invention may also be directed to an antigen recognizing construct that binds the peptide VVGAVGVGK (SEQ ID NO: 1) with determinable affinity, wherein the antigen recognizing construct comprises a complementary determining region 3 (CDR3) of the α-chain having at least 90%, at least 91%, at least 92%, at least 93%, at least 94%, at least 95%, at least 96%, at least 97%, at least 98%, or at least 99% sequence identity to an amino acid sequence of SEQ ID NO: 10, and/or wherein the antigen recognizing construct comprises a complementary determining region 3 (CDR3) of the β-chain having at least 90%, at least 91%, at least 92%, at least 93%, at least 94%, at least 95%, at least 96%, at least 97%, at least 98%, or at least 99% sequence identity to an amino acid sequence of SEQ ID NO: 13.

The peptide VVGAVGVGK (SEQ ID NO: 1) is a mutated KRAS peptide, namely of the region of amino acids 8-16 of KRAS with the point mutation G12V (KRAS^{G12V}) Thus, the terms “mutated KRAS” or “mutated target”, as used herein, specifically may mean the G12V point mutant of amino acids 8-16 of KRAS. The peptide VVGAVGVGK (SEQ ID NO: 1) is thus a respective mutated KRAS epitope. Thus, the antigen recognizing constructs or the TCRs according to the invention have the ability to bind the mutated KRAS epitope/peptide of VVGAVGVGK (SEQ ID NO: 1) (KRAS^{G12V}) with determinable affinity.

The term “with determinable affinity” as used herein means that the antigen recognizing construct or T cell receptor according to the invention may bind the respective peptide i.e. with an affinity constant of at least 10⁴ M⁻¹. The term “affinity constant” as used herein means the equilibrium association constant Ka. Lower affinities, as a rule, can no longer be recorded exactly with the customary measuring methods and are therefore of minor importance for practical applications. The antigen recognizing construct or T cell receptor may, for example, bind the respective peptide with an affinity constant of at least 10⁴ M⁻¹, corresponding to an equilibrium dissociation constant Kd of 100 μM. The binding affinity of the antigen recognizing construct or T cell receptor to the respective peptide can be determined by a person skilled in the art using a large number of methods, for example using the method of fluorescence titration, using competition ELISA or using the surface plasmon resonance technique.

The term “epitope” or “antigenic epitope” includes any molecule, structure, amino acid sequence or protein determinant that is recognized and specifically bound by a cognate binding molecule, such as an immunoglobulin, T cell receptor (TCR), chimeric antigen receptor, or other binding molecule, domain or protein. Epitopic determinants generally contain chemically active surface groupings of molecules, such as amino acids or sugar side chains, and can have specific three dimensional structural characteristics, as well as specific charge characteristics.

The antigen recognizing construct according to the first aspect of the invention may be an antibody, or fragment thereof, or a T cell receptor (TCR), or fragment thereof.

Thus, it is within the scope of the first aspect of the invention that the antigen recognizing construct is an antibody, or a fragment thereof. The term “antibody” in its various grammatical forms is used herein to refer to immunoglobulin molecules and immunologically active portions of immunoglobulin molecules, i.e., molecules that contain an antibody combining site or a paratope. Such molecules are also referred to as “antigen binding fragments” of immunoglobulin molecules. The invention further provides an antibody, or antigen binding portion thereof, which specifically binds to the antigens described herein. The antibody can be any type of immunoglobulin that is known in the art. For instance, the antibody can be of any isotype, e.g., IgA, IgD, IgE, IgG, IgM, etc. The antibody can be monoclonal or polyclonal. The antibody can be a naturally-occurring antibody, e.g., an antibody isolated and/or purified from a mammal, e.g., mouse, rabbit, goat, horse, chicken, hamster, human, etc. Alternatively, the antibody can be a genetically-engineered antibody, e.g., a humanized antibody or a chimeric antibody. The antibody can be in monomeric or polymeric form.

A “T cell” or “T lymphocyte” is an immune system cell that matures in the thymus and produces T cell receptors (TCR). T cells can be naive (“T_{N}”; not exposed to antigen; increased expression of CD62L, CCR7, CD28, CD3, CD127, and CD45RA, and decreased or no expression of CD45RO as compared to T_{CM}), memory T cells (T_{M}) (antigen experienced and long-lived), including stem cell memory T cells, and effector cells (antigen-experienced, cytotoxic). T_{M} can be further divided into subsets of central memory T cells (T_{EM}, expresses CD62L, CCR7, CD28, CD95, CD45RO, and CD127) and effector memory T cells (T_{EM} express CD45RO, decreased expression of CD62L, CCR7, CD28, and CD45RA). Effector T cells (T_{E}) refers to antigen-experienced CD8+ cytotoxic T lymphocytes that express CD45RA, have decreased expression of CD62L, CCR7, and CD28 as compared to T_{CM}, and are positive for granzyme and perforin. CD4+ cells influence the activity of other immune cells by releasing cytokines. CD4+ T cells can activate and suppress an adaptive immune response, and which of those two functions is induced will depend on the presence of other cells and signals. T cells can be collected using known techniques, and the various subpopulations or combinations thereof can be enriched or depleted by known techniques, such as by affinity binding to antibodies, flow cytometry, or immunomagnetic selection. Other exemplary T cells include regulatory T cells, such as CD4+CD25+(Foxp3+) regulatory T cells and Treg17 cells, as well as T1, Th3, and Qa-1 restricted T cells.

“T cell receptor” (TCR) refers to an immunoglobulin superfamily member having a variable binding domain, a constant domain, a transmembrane region, and a short cytoplasmic tail; see, e. g., Janeway el al., Immunobiology: The Immune System in Health and Disease, 3rd Ed., Current Biology Publications, p. 433, 1997) capable of specifically binding to an antigen peptide bound to an MHC receptor. A TCR can be found on the surface of a cell or in soluble form and generally is comprised of a heterodimer having α and β chains (also known as TCR α and TCR β, respectively), or γ and δ chains (also known as TCRγ and TCRδ, respectively). A polynucleotide encoding a binding protein of this disclosure, e.g., a TCR, can be codon optimized to enhance expression in a particular host cell, such, for example, as a cell of the immune system, a hematopoietic stem cell, a T cell, a primary T cell, a T cell line, a NK cell, or a natural killer T cell (Scholten el al., Clin. Immunol. 119:135, 2006).

The terms “complementarity determining region” and “CDR,” are synonymous with “hypervariable region” or “HVR” and are known in the art to refer to sequences of amino acids within immunoglobulin or TCR variable regions, which confer antigen specificity and/or binding affinity and are separated from one another in primary amino acid sequence by framework regions. In general, there are three CDRs in each TCR α-chain variable region (αCDR1, αCDR2, αCDR3) and three CDRs in each TCR β-chain variable region (βCDR1, βCDR2, βCDR3). In TCRs, CDR3 is thought to be the main CDR responsible for recognizing processed antigen. In general, CDR1 and CDR2 interact mainly or exclusively with the MHC.

The antigen recognizing construct may specifically bind the peptide VVGAVGVGK (SEQ ID NO: 1) with a half-maximal effective concentration, EC₅₀, of less than about 1×10⁻⁶ M. In illustrated examples, the antigen recognizing construct may specifically bind the peptide VVGAVGVGK (SEQ ID NO: 1) with an EC₅₀ of less than about 1×10⁻⁸ M or of less than about 2×10⁻⁸ M or of less than about 6×10⁻⁸ M. The EC₅₀ value may be determined by the procedure as carried out in Example 2 as described herein.

The term “specifically bind(s)”, as used herein, refers to an association or union of an antigen recognizing construct (e.g., TCR receptor) to a target peptide with an affinity or Kₐ (i.e., an equilibrium association constant of a particular binding interaction with units of 1/M) equal to or greater than 10⁴ M⁻¹ (which equals the ratio of the on-rate [kₒₙ] to the off-rate [k_{off}] for this association reaction), while not significantly associating or uniting with any other molecules or components in a sample.

The antigen recognizing construct according to the first aspect of the invention, which binds the peptide VVGAVGVGK (SEQ ID NO: 1), may be presented by a MHC I molecule. For example, the MHC I molecule may be an HLA-A molecule. The HLA-A molecule may be, for example, HLA-A*11:01. An exemplary amino acid sequence of HLA-A*11:01 is IMGT/HLA accession number HLA00043 (see https://www.ebi.ac.uk/ipd/imgt/hla/alleles/allele/?accession=HLA00043).

There are two classes of MHC molecules, MHC class I and MHC class II. Complexes of peptide and MHC class I are recognized by CD8-positive T cells bearing the appropriate T-cell receptor (TCR), whereas complexes of peptide and MHC class II molecules are recognized by CD4-positive T cells bearing the appropriate TCR. Since both types of responses, CD8 and CD4 T cell dependent responses, contribute jointly and synergistically to the anti-tumor effect, the identification and characterization of tumor-associated and tumor-specific antigens and corresponding T cell receptors is important in the development of cancer immunotherapies, such as vaccines and cell therapies.

It is within the scope of the first aspect of the invention that the antigen recognizing construct may bind the peptide with an antigenic specificity of at least 80%. It is within the scope of the first aspect of the invention that the antigen recognizing construct may bind the peptide with an antigenic specificity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%.

The term “antigenic specificity,” as used herein, means that the antigen recognizing construct can specifically bind to and immunologically recognize mutated target, e.g., mutated KRAS. It may mean the ability to recognize an antigen specifically as a unique molecular entity and distinguish it from another with exquisite precision. For example, an antigen-recognizing construct may be considered to have “antigenic specificity” for mutated target if about 1×10⁴ to about 1×10⁵ T cells expressing the antigen recognizing construct secrete at least about 200 pg/mL or more (e.g., 200 pg/mL or more, 300 pg/mL or more, 400 pg/mL or more, 500 pg/mL or more, 600 pg/mL or more, 700 pg/mL or more, 1000 pg/mL or more, 5,000 pg/mL or more, 7,000 pg/mL or more, 10,000 pg/mL or more, 20,000 pg/mL or more, or a range defined by any two of the foregoing values) of IFNγ upon co-culture with (a) antigen-negative HLA-A11:01 target cells pulsed with a low concentration of mutated target peptide (e.g., about 0.05 ng/mL to about 5 ng/mL, 0.05 ng/mL, 0.1 ng/mL, 0.5 ng/mL, 1 ng/mL, 5 ng/mL, or a range defined by any two of the foregoing values) or (b) antigen-negative HLA-A11:01 target cells into which a nucleotide sequence encoding the mutated target has been introduced such that the target cell expresses the mutated target. Cells expressing the inventive antigen recognizing constructs may also secrete IFNγ upon co-culture with antigen-negative HLA-A11:01 target cells pulsed with higher concentrations of mutated target peptide.

Any antibody molecule that specifically binds KRAS can be used herein. The antibody molecule can, for example, be a polyclonal antibody, a monoclonal antibody, or fragments typically derived from monoclonal antibodies, such as divalent antigen binding antibody fragments, or monovalent antigen binding antibody fragments. Examples of suitable divalent antibody fragments are (Fab)₂′-fragments, divalent single-chain Fv fragments or divalent single domain camelid antibodies (also knowns as nanobodies) that can be obtained by producing such single domain camelid antibodies as fusion proteins with a linker between the two single domain camelid antibodies. Examples of suitable monovalent antibody fragments include, but are not limited to, a Fab fragment, a Fv fragment, or a single-chain Fv fragment (scFv).

Specifically, as mentioned above, the antigen recognizing construct may be a T cell receptor (TCR). Such a TCR may comprise a complementary determining region 1 (CDR1) of the α-chain having at least 90%, at least 91%, at least 92%, at least 93%, at least 94%, at least 95%, at least 96%, at least 97%, at least 98% or at least 99% sequence identity to an amino acid sequence selected from the group consisting of SEQ ID NOs: 8, 14, 2, 20, and 26, and/or wherein the T cell receptor comprises a complementary determining region 1 (CDR1) of the β-chain having at least 90%, at least 91%, at least 92%, at least 93%, at least 94%, at least 95%, at least 96%, at least 97%, at least 98% or at least 99% sequence identity to an amino acid sequence selected from the group consisting of SEQ ID NOs: 11, 17, 5, 23, and 29. Further, the T cell receptor may comprise a complementary determining region 1 (CDR1) of the α-chain having at least 90%, at least 91%, at least 92%, at least 93%, at least 94%, at least 95%, at least 96%, at least 97%, at least 98% or at least 99% sequence identity to an amino acid sequence selected from the group consisting of SEQ ID NO: 8, and/or wherein the T cell receptor comprises a complementary determining region 1 (CDR1) of the β-chain having at least 90%, at least 91%, at least 92%, at least 93%, at least 94%, at least 95%, at least 96%, at least 97%, at least 98% or at least 99% sequence identity to an amino acid sequence of SEQ ID NO: 11.

Further, the T cell receptor may comprise a complementary determining region 2 (CDR2) of the α-chain having at least 90%, at least 91%, at least 92%, at least 93%, at least 94%, at least 95%, at least 96%, at least 97%, at least 98% or at least 99% sequence identity to an amino acid sequence selected from the group consisting of SEQ ID NOs: 9, 15, 3, 21, and 27, and/or that the T cell receptor may comprise a complementary determining region 2 (CDR2) of the β-chain having at least 90%, at least 91%, at least 92%, at least 93%, at least 94%, at least 95%, at least 96%, at least 97%, at least 98% or at least 99% sequence identity to an amino acid sequence selected from the group consisting of SEQ ID NOs: 12, 18, 6, 24, and 30. Further, the T cell receptor may comprise a complementary determining region 2 (CDR2) of the α-chain having at least 90%, at least 91%, at least 92%, at least 93%, at least 94%, at least 95%, at least 96%, at least 97%, at least 98% or at least 99% sequence identity to an amino acid sequence of SEQ ID NO: 9, and/or that the T cell receptor may comprise a complementary determining region 2 (CDR2) of the β-chain having at least 90%, at least 91%, at least 92%, at least 93%, at least 94%, at least 95%, at least 96%, at least 97%, at least 98% or at least 99% sequence identity to an amino acid sequence of SEQ ID NO: 12.

It is also comprised in the first aspect of the invention that the T cell receptor may comprise a complementary determining region 3 (CDR3) of the α-chain having at least 95%, at least 96%, at least 97%, at least 98% or at least 99% sequence identity to an amino acid sequence selected from the group consisting of SEQ ID NOs: 10, 16, 4, 22, and 28, and/or that the T cell receptor may comprise a complementary determining region 3 (CDR3) of the β-chain having at least 95%, at least 96%, at least 97%, at least 98% or at least 99% sequence identity to an amino acid sequence selected from the group consisting of SEQ ID NOs: 13, 19, 7, 25, and 31. It is also comprised in the first aspect of the invention that the T cell receptor may comprise a complementary determining region 3 (CDR3) of the α-chain having at least 95%, at least 96%, at least 97%, at least 98% or at least 99% sequence identity to an amino acid sequence of SEQ ID NO: 10, and/or that the T cell receptor may comprise a complementary determining region 3 (CDR3) of the β-chain having at least 95%, at least 96%, at least 97%, at least 98% or at least 99% sequence identity to an amino acid sequence of SEQ ID NO: 13. The T cell receptor may comprise a complementary determining region 1 (CDR1) of the α-chain having at least 95%, at least 96%, at least 97%, at least 98% or at least 99% sequence identity to an amino acid sequence selected from the group consisting of SEQ ID NOs: 8, 14, 2, 20, and 26, and/or the T cell receptor may comprise a complementary determining region 1 (CDR1) of the β-chain having at least 95%, at least 96%, at least 97%, at least 98% or at least 99% sequence identity to an amino acid sequence selected from the group consisting of SEQ ID NOs: 11, 17, 5, 23, and 29. The T cell receptor may comprise a complementary determining region 1 (CDR1) of the α-chain having at least 95%, at least 96%, at least 97%, at least 98% or at least 99% sequence identity to an amino acid sequence of SEQ ID NO: 8, and/or the T cell receptor may comprise a complementary determining region 1 (CDR1) of the β-chain having at least 95%, at least 96%, at least 97%, at least 98% or at least 99% sequence identity to an amino acid sequence of SEQ ID NO: 11. The T cell receptor may comprise a complementary determining region 2 (CDR2) of the α-chain having at least 95%, at least 96%, at least 97%, at least 98% or at least 99% sequence identity to an amino acid sequence selected from the group consisting of SEQ ID NOs: 9, 15, 3, 21, and 27, and/or the T cell receptor may comprise a complementary determining region 2 (CDR2) of the β-chain having at least 95%, at least 96%, at least 97%, at least 98% or at least 99% sequence identity to an amino acid sequence selected from the group consisting of SEQ ID NOs: 12, 18, 6, 24, and 30.

The T cell receptor according to the first aspect of the invention may comprise
(a) an α-chain CDR1 comprising or consisting of the amino acid sequence of SEQ ID NO: 8,
an α-chain CDR2 comprising or consisting of the amino acid sequence of SEQ ID NO: 9, and
an α-chain CDR3 comprising or consisting of the amino acid sequence of SEQ ID NO: 10; or
(b) an α-chain CDR1 comprising or consisting of the amino acid sequence of SEQ ID NO: 14,
an α-chain CDR2 comprising or consisting of the amino acid sequence of SEQ ID NO: 15, and
an α-chain CDR3 comprising or consisting of the amino acid sequence of SEQ ID NO: 16; or
(c) an α-chain CDR1 comprising or consisting of the amino acid sequence of SEQ ID NO: 2,
an α-chain CDR2 comprising or consisting of the amino acid sequence of SEQ ID NO: 3, and
an α-chain CDR3 comprising or consisting of the amino acid sequence of SEQ ID NO: 4; or
(d) an α-chain CDR1 comprising or consisting of the amino acid sequence of SEQ ID NO: 20,
an α-chain CDR2 comprising or consisting of the amino acid sequence of SEQ ID NO: 21, and
an α-chain CDR3 comprising or consisting of the amino acid sequence of SEQ ID NO: 22; or
(e) an α-chain CDR1 comprising or consisting of the amino acid sequence of SEQ ID NO: 26,
an α-chain CDR2 comprising or consisting of the amino acid sequence of SEQ ID NO: 27, and
an α-chain CDR3 comprising or consisting of the amino acid sequence of SEQ ID NO: 28.

The T cell receptor according to the first aspect of the invention may comprise
(a) a β-chain CDR1 comprising or consisting of the amino acid sequence of SEQ ID NO: 11,
a β-chain CDR2 comprising or consisting of the amino acid sequence of SEQ ID NO: 12, and
a β-chain CDR3 comprising or consisting of the amino acid sequence of SEQ ID NO: 13; or
(b) a β-chain CDR1 comprising or consisting of the amino acid sequence of SEQ ID NO: 17,
a β-chain CDR2 comprising or consisting of the amino acid sequence of SEQ ID NO: 18, and
a β-chain CDR3 comprising or consisting of the amino acid sequence of SEQ ID NO: 19; or
(c) a β-chain CDR1 comprising or consisting of the amino acid sequence of SEQ ID NO: 5,
a β-chain CDR2 comprising or consisting of the amino acid sequence of SEQ ID NO: 6, and
a β-chain CDR3 comprising or consisting of the amino acid sequence of SEQ ID NO: 7; or
(d) a β-chain CDR1 comprising or consisting of the amino acid sequence of SEQ ID NO: 23,
a β-chain CDR2 comprising or consisting of the amino acid sequence of SEQ ID NO: 24, and
a β-chain CDR3 comprising or consisting of the amino acid sequence of SEQ ID NO: 25; or
(e) a β-chain CDR1 comprising or consisting of the amino acid sequence of SEQ ID NO: 29,
a β-chain CDR2 comprising or consisting of the amino acid sequence of SEQ ID NO: 30, and
a β-chain CDR3 comprising or consisting of the amino acid sequence of SEQ ID NO: 31.

The T cell receptor according to the first aspect of the invention may comprise
(a) an α-chain CDR1 comprising or consisting of the amino acid sequence of SEQ ID NO: 8,
an α-chain CDR2 comprising or consisting of the amino acid sequence of SEQ ID NO: 9,
an α-chain CDR3 comprising or consisting of the amino acid sequence of SEQ ID NO: 10;
a β-chain CDR1 comprising or consisting of the amino acid sequence of SEQ ID NO: 11,
a β-chain CDR2 comprising or consisting of the amino acid sequence of SEQ ID NO: 12, and
a β-chain CDR3 comprising or consisting of the amino acid sequence of SEQ ID NO: 13; or
(b) an α-chain CDR1 comprising or consisting of the amino acid sequence of SEQ ID NO: 14,
an α-chain CDR2 comprising or consisting of the amino acid sequence of SEQ ID NO: 15,
an α-chain CDR3 comprising or consisting of the amino acid sequence of SEQ ID NO: 16;
a β-chain CDR1 comprising or consisting of the amino acid sequence of SEQ ID NO: 17,
a β-chain CDR2 comprising or consisting of the amino acid sequence of SEQ ID NO: 18, and
a β-chain CDR3 comprising or consisting of the amino acid sequence of SEQ ID NO: 19; or
(c) an α-chain CDR1 comprising or consisting of the amino acid sequence of SEQ ID NO: 2,
an α-chain CDR2 comprising or consisting of the amino acid sequence of SEQ ID NO: 3,
an α-chain CDR3 comprising or consisting of the amino acid sequence of SEQ ID NO: 4,
a β-chain CDR1 comprising or consisting of the amino acid sequence of SEQ ID NO: 5,
a β-chain CDR2 comprising or consisting of the amino acid sequence of SEQ ID NO: 6, and
a β-chain CDR3 comprising or consisting of the amino acid sequence of SEQ ID NO: 7; or
(d) an α-chain CDR1 comprising or consisting of the amino acid sequence of SEQ ID NO: 20,
an α-chain CDR2 comprising or consisting of the amino acid sequence of SEQ ID NO: 21,
an α-chain CDR3 comprising or consisting of the amino acid sequence of SEQ ID NO: 22;
a β-chain CDR1 comprising or consisting of the amino acid sequence of SEQ ID NO: 23,
a β-chain CDR2 comprising or consisting of the amino acid sequence of SEQ ID NO: 24, and
a β-chain CDR3 comprising or consisting of the amino acid sequence of SEQ ID NO: 25; or
(e) an α-chain CDR1 comprising or consisting of the amino acid sequence of SEQ ID NO: 26,
an α-chain CDR2 comprising or consisting of the amino acid sequence of SEQ ID NO: 27,
an α-chain CDR3 comprising or consisting of the amino acid sequence of SEQ ID NO: 28;
a β-chain CDR1 comprising or consisting of the amino acid sequence of SEQ ID NO: 29,
a β-chain CDR2 comprising or consisting of the amino acid sequence of SEQ ID NO: 30, and
a β-chain CDR3 comprising or consisting of the amino acid sequence of SEQ ID NO: 31.

The T cell receptor according to the first aspect of the invention may comprise
(a) an α chain comprising CDR sequences comprising amino acid sequences of SEQ ID NOs: 8, 9, and 10, and a β chain comprising CDR sequences comprising amino acid sequences of SEQ ID NOs: 11, 12, and 13; or
(b) an α chain comprising CDR sequences comprising amino acid sequences of SEQ ID NOs: 14, 15, and 16, and a β chain comprising CDR sequences comprising amino acid sequences of SEQ ID NOs: 17, 18, and 19; or
(c) an α chain comprising CDR sequences comprising amino acid sequences of SEQ ID NOs: 2, 3, and 4, and a β chain comprising CDR sequences comprising amino acid sequences of SEQ ID NOs: 5, 6, and 7; or
(d) an α chain comprising CDR sequences comprising amino acid sequences of SEQ ID NOs: 20, 21, and 22, and a β chain comprising CDR sequences comprising amino acid sequences of SEQ ID NOs: 23, 24, and 25; or
(e) an α chain comprising CDR sequences comprising amino acid sequences of SEQ ID NO: 26, 27, and 28, and a β chain comprising CDR sequences comprising amino acid sequences of SEQ ID NOs: 29, 30, and 31.

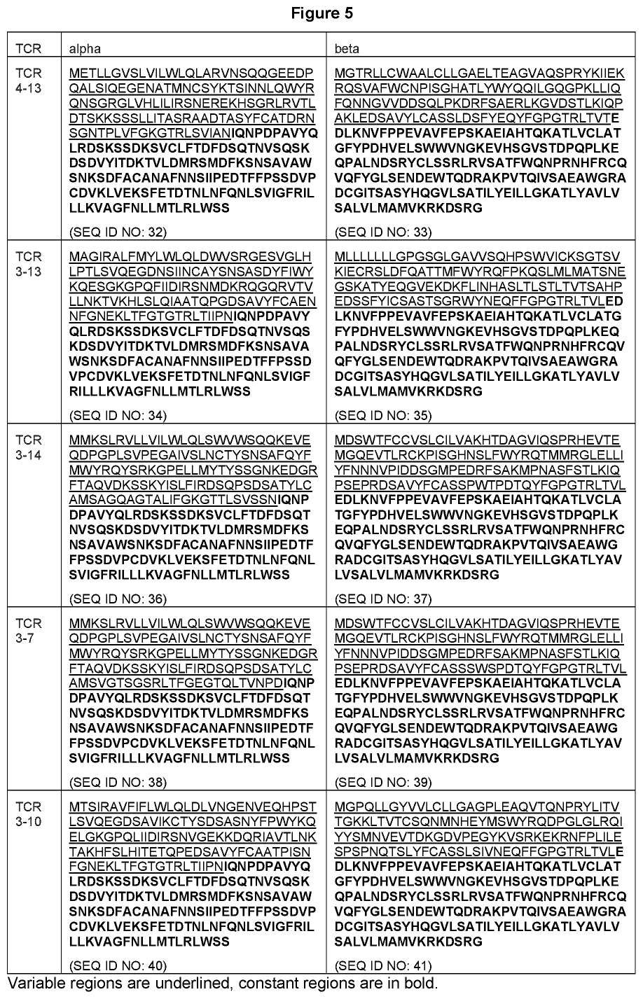

The T cell receptor according to the first aspect of the invention may comprise
(a) an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the variable domain of SEQ ID NO: 32, and/or an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the variable domain of SEQ ID NO: 33; or
(b) an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the variable domain of SEQ ID NO: 34, and/or an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the variable domain of SEQ ID NO: 35; or
(c) an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the variable domain of SEQ ID NO: 36, and/or an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the variable domain of SEQ ID NO: 37; or
(d) an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the variable domain of SEQ ID NO: 38, and/or an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the variable domain of SEQ ID NO: 39; or
(e) an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the variable domain of SEQ ID NO: 40, and/or an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the variable domain of SEQ ID NO: 41. The variable domains or regions of the respective SEQ ID NOs are depicted herein in Table 2 and FIG. 5 by underlinements.

The T cell receptor according to the first aspect of the invention may comprise
(a) an α chain comprising a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the amino acid sequence of SEQ ID NO: 32, and/or a β chain comprising a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the amino acid sequence of SEQ ID NO: 33; or
(b) an α chain comprising a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the amino acid sequence of SEQ ID NO: 34, and/or a β chain comprising a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the amino acid sequence of SEQ ID NO: 35; or
(c) an α chain comprising a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the amino acid sequence of SEQ ID NO: 36, and/or a β chain comprising a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the amino acid sequence of SEQ ID NO: 37; or
(d) an α chain comprising a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the amino acid sequence of SEQ ID NO: 38, and/or a β chain comprising a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the amino acid sequence of SEQ ID NO: 39; or
(e) an α chain comprising a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the amino acid sequence of SEQ ID NO: 40, and/or a β chain comprising a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the amino acid sequence of SEQ ID NO: 41.

In yet a further aspect, the invention provides a T cell receptor (TCR) having antigenic specificity for mutated KRAS, wherein the TCR comprises:
(a) an α-chain CDR1 comprising or consisting of the amino acid sequence of SEQ ID NO: 8, an α-chain CDR2 comprising or consisting of the amino acid sequence of SEQ ID NO: 9, and
an α-chain CDR3 comprising or consisting of the amino acid sequence of SEQ ID NO: 10; or
(b) an α-chain CDR1 comprising or consisting of the amino acid sequence of SEQ ID NO: 14,
an α-chain CDR2 comprising or consisting of the amino acid sequence of SEQ ID NO: 15, and
an α-chain CDR3 comprising or consisting of the amino acid sequence of SEQ ID NO: 16; or
(c) an α-chain CDR1 comprising or consisting of the amino acid sequence of SEQ ID NO: 2,
an α-chain CDR2 comprising or consisting of the amino acid sequence of SEQ ID NO: 3, and
an α-chain CDR3 comprising or consisting of the amino acid sequence of SEQ ID NO: 4; or
(d) an α-chain CDR1 comprising or consisting of the amino acid sequence of SEQ ID NO: 20,
an α-chain CDR2 comprising or consisting of the amino acid sequence of SEQ ID NO: 21, and
an α-chain CDR3 comprising or consisting of the amino acid sequence of SEQ ID NO: 22; or
(e) an α-chain CDR1 comprising or consisting of the amino acid sequence of SEQ ID NO: 26,
an α-chain CDR2 comprising or consisting of the amino acid sequence of SEQ ID NO: 27, and
an α-chain CDR3 comprising or consisting of the amino acid sequence of SEQ ID NO: 28.

In yet a further aspect, the invention provides a T cell receptor (TCR) having antigenic specificity for mutated KRAS, wherein the TCR comprises:
(a) a β-chain CDR1 comprising or consisting of the amino acid sequence of SEQ ID NO: 11,
a β-chain CDR2 comprising or consisting of the amino acid sequence of SEQ ID NO: 12, and
a β-chain CDR3 comprising or consisting of the amino acid sequence of SEQ ID NO: 13; or
(b) a β-chain CDR1 comprising or consisting of the amino acid sequence of SEQ ID NO: 17,
a β-chain CDR2 comprising or consisting of the amino acid sequence of SEQ ID NO: 18, and
a β-chain CDR3 comprising or consisting of the amino acid sequence of SEQ ID NO: 19; or
(c) a β-chain CDR1 comprising or consisting of the amino acid sequence of SEQ ID NO: 5,
a β-chain CDR2 comprising or consisting of the amino acid sequence of SEQ ID NO: 6, and
a β-chain CDR3 comprising or consisting of the amino acid sequence of SEQ ID NO: 7; or
(d) a β-chain CDR1 comprising or consisting of the amino acid sequence of SEQ ID NO: 23,
a β-chain CDR2 comprising or consisting of the amino acid sequence of SEQ ID NO: 24, and
a β-chain CDR3 comprising or consisting of the amino acid sequence of SEQ ID NO: 25; or
(e) a β-chain CDR1 comprising or consisting of the amino acid sequence of SEQ ID NO: 29,
a β-chain CDR2 comprising or consisting of the amino acid sequence of SEQ ID NO: 30, and
a β-chain CDR3 comprising or consisting of the amino acid sequence of SEQ ID NO: 31.

In a second aspect, the invention provides a T cell receptor (TCR) having antigenic specificity for mutated KRAS, wherein the TCR may comprise:
(a) an α-chain CDR1 comprising or consisting of the amino acid sequence of SEQ ID NO: 8,
an α-chain CDR2 comprising or consisting of the amino acid sequence of SEQ ID NO: 9,
an α-chain CDR3 comprising or consisting of the amino acid sequence of SEQ ID NO: 10;
a β-chain CDR1 comprising or consisting of the amino acid sequence of SEQ ID NO: 11,
a β-chain CDR2 comprising or consisting of the amino acid sequence of SEQ ID NO: 12, and
a β-chain CDR3 comprising or consisting of the amino acid sequence of SEQ ID NO: 13; or
(b) an α-chain CDR1 comprising or consisting of the amino acid sequence of SEQ ID NO: 14,
an α-chain CDR2 comprising or consisting of the amino acid sequence of SEQ ID NO: 15,
an α-chain CDR3 comprising or consisting of the amino acid sequence of SEQ ID NO: 16;
a β-chain CDR1 comprising or consisting of the amino acid sequence of SEQ ID NO: 17,
a β-chain CDR2 comprising or consisting of the amino acid sequence of SEQ ID NO: 18, and
a β-chain CDR3 comprising or consisting of the amino acid sequence of SEQ ID NO: 19; or
(c) an α-chain CDR1 comprising or consisting of the amino acid sequence of SEQ ID NO: 2,
an α-chain CDR2 comprising or consisting of the amino acid sequence of SEQ ID NO: 3,
an α-chain CDR3 comprising or consisting of the amino acid sequence of SEQ ID NO: 4,
a β-chain CDR1 comprising or consisting of the amino acid sequence of SEQ ID NO: 5,
a β-chain CDR2 comprising or consisting of the amino acid sequence of SEQ ID NO: 6, and
a β-chain CDR3 comprising or consisting of the amino acid sequence of SEQ ID NO: 7; or
(d) an α-chain CDR1 comprising or consisting of the amino acid sequence of SEQ ID NO: 20,
an α-chain CDR2 comprising or consisting of the amino acid sequence of SEQ ID NO: 21,
an α-chain CDR3 comprising or consisting of the amino acid sequence of SEQ ID NO: 22;
a β-chain CDR1 comprising or consisting of the amino acid sequence of SEQ ID NO: 23,
a β-chain CDR2 comprising or consisting of the amino acid sequence of SEQ ID NO: 24, and
a β-chain CDR3 comprising or consisting of the amino acid sequence of SEQ ID NO: 25; or
(e) an α-chain CDR1 comprising or consisting of the amino acid sequence of SEQ ID NO: 26,
an α-chain CDR2 comprising or consisting of the amino acid sequence of SEQ ID NO: 27,
an α-chain CDR3 comprising or consisting of the amino acid sequence of SEQ ID NO: 28;
a β-chain CDR1 comprising or consisting of the amino acid sequence of SEQ ID NO: 29,
a β-chain CDR2 comprising or consisting of the amino acid sequence of SEQ ID NO: 30, and
a β-chain CDR3 comprising or consisting of the amino acid sequence of SEQ ID NO: 31.

In a second aspect, the invention provides a T cell receptor (TCR) having antigenic specificity for mutated KRAS, wherein the TCR may comprise:
(a) an α chain comprising CDR sequences comprising amino acid sequences of SEQ ID NOs: 8, 9, and 10, and a β chain comprising CDR sequences comprising amino acid sequences of SEQ ID NOs: 11, 12, and 13; or
(b) an α chain comprising CDR sequences comprising amino acid sequences of SEQ ID NOs: 14, 15, and 16, and a β chain comprising CDR sequences comprising amino acid sequences of SEQ ID NOs: 17, 18, and 19; or
(c) an α chain comprising CDR sequences comprising amino acid sequences of SEQ ID NOs: 2, 3, and 4, and a β chain comprising CDR sequences comprising amino acid sequences of SEQ ID NOs: 5, 6, and 7; or
(d) an α chain comprising CDR sequences comprising amino acid sequences of SEQ ID NOs: 20, 21, and 22, and a β chain comprising CDR sequences comprising amino acid sequences of SEQ ID NOs: 23, 24, and 25; or
(e) an α chain comprising CDR sequences comprising amino acid sequences of SEQ ID NO: 26, 27, and 28, and a β chain comprising CDR sequences comprising amino acid sequences of SEQ ID NOs: 29, 30, and 31.

In a second aspect, the invention provides a T cell receptor (TCR) having antigenic specificity for mutated KRAS, wherein the TCR may comprise:
(a) an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the variable domain of SEQ ID NO: 32, and/or an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the variable domain of SEQ ID NO: 33; or
(b) an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the variable domain of SEQ ID NO: 34, and/or an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the variable domain of SEQ ID NO: 35; or
(c) an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the variable domain of SEQ ID NO: 36, and/or an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the variable domain of SEQ ID NO: 37; or
(d) an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the variable domain of SEQ ID NO: 38, and/or an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the variable domain of SEQ ID NO: 39; or
(e) an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the variable domain of SEQ ID NO: 40, and/or an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the variable domain of SEQ ID NO: 41. The variable domains or regions of the respective SEQ ID NOs are depicted herein in Table 2 and FIG. 5 by underlinements.

In a second aspect, the invention provides a T cell receptor (TCR) having antigenic specificity for mutated KRAS, wherein the TCR may comprise: (a) an α chain comprising a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the amino acid sequence of SEQ ID NO: 32, and/or a β chain comprising a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the amino acid sequence of SEQ ID NO: 33; or
(b) an α chain comprising a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the amino acid sequence of SEQ ID NO: 34, and/or a β chain comprising a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the amino acid sequence of SEQ ID NO: 35; or
(c) an α chain comprising a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the amino acid sequence of SEQ ID NO: 36, and/or a β chain comprising a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the amino acid sequence of SEQ ID NO: 37; or
(d) an α chain comprising a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the amino acid sequence of SEQ ID NO: 38, and/or a β chain comprising a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the amino acid sequence of SEQ ID NO: 39; or
(e) an α chain comprising a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the amino acid sequence of SEQ ID NO: 40, and/or a β chain comprising a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the amino acid sequence of SEQ ID NO: 41.

Included in the scope of the invention are TCRs comprising at least one α and/or β TCR chain, wherein said α TCR chain comprises a sequence having at least 50%, at least 60%, at least 70%, at least 80%, at least 90%, at least 91%, at least 92%, at least 93%, at least 94%, at least 95%, at least 96%, at least 97%, at least 98%, at least 99% or 100% sequence identity to an amino acid sequence of SEQ ID NOs: 32, 34, 36, 38, or 40, and/or wherein said β TCR chain comprises a sequence having at least 50%, at least 60%, at least 70%, at least 80%, at least 90%, at least 91%, at least 92%, at least 93%, at least 94%, at least 95%, at least 96%, at least 97%, at least 98%, at least 99% or 100% sequence identity to an amino acid sequence of SEQ ID NOs: 33, 35, 37, 39, or 41.

Included in the scope of the invention are functional variants of the inventive antigen recognizing constructs or TCRs described herein. The term “functional variant,” as used herein, refers to an antigen recognizing construct or TCR having substantial or significant sequence identity or similarity to a parent antigen recognizing construct or TCR, which functional variant retains the biological activity of the antigen recognizing construct or TCR of which it is a variant. Functional variants encompass, for example, those variants of the antigen recognizing construct or TCR described herein (the parent antigen recognizing construct or TCR) that retain the ability to specifically bind to mutated target, e.g., mutated KRAS, for which the parent antigen recognizing construct or TCR has antigenic specificity to a similar extent, the same extent, or to a higher extent, as the parent antigen recognizing construct or TCR. In reference to the parent antigen recognizing construct or TCR, the functional variant can, for instance, be at least about 30%, 50%, 75%, 80%, 90%, 95%, 96%, 97%, 98%, 99% or more identical in amino acid sequence to the parent antigen recognizing construct or TCR.

The term “sequence identity” or “identity” as used in the present invention means the percentage of pair-wise identical residues, following homology alignment of a sequence of a polypeptide of the present invention with a sequence in question, with respect to the number of residues in the longer of these two sequences.

The percentage of sequence homology or sequence identity can, for example, be determined herein using the program BLASTP, version blastp 2.2.5 (Nov. 16, 2002; cf. Altschul, S. F. et al. (1997) Nucl. Acids Res. 25, 3389-3402). The percentage of homology is based on the alignment of the entire polypeptide sequences (matrix: BLOSUM 62; gap costs: 11.1; cutoff value set to 10⁻³) including the respective sequences. It is calculated as the percentage of numbers of “positives” (homologous amino acids) indicated as result in the BLASTP program output divided by the total number of amino acids selected by the program for the alignment.

The functional variant can, for example, comprise the amino acid sequence of the parent antigen recognizing construct or TCR with at least one conservative amino acid substitution. Conservative amino acid substitutions are known in the art, and include amino acid substitutions in which one amino acid having certain physical and/or chemical properties is exchanged for another amino acid that has the same chemical or physical properties. For instance, the conservative amino acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, lie, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc.

The term “KRAS” means Kirsten rat sarcoma virus and its protein sequence is e.g. deposited under the UniProtKB accession number P01116 (RASK HUMAN).

Reverting to the aspects of the invention, in the third aspect the invention is directed to a nucleic acid sequence encoding the antigen recognizing construct or the T cell receptor according to the first or second aspect of the invention.

The term “nucleic acid”, as used herein, includes “polynucleotide”, “oligonucleotide” and “nucleic acid molecule”, and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide.

In the fourth aspect, the invention is directed to a vector comprising the nucleic acid sequence according to the third aspect of the invention.

In the context of the present invention, the term “vector” encompasses a DNA molecule, such as a plasmid, bacteriophage, phagemid, virus or other vehicle, which contains one or more heterologous or recombinant nucleotide sequences (e.g., an above-described nucleic acid molecule of the invention, under the control of a functional promoter and, possibly, also an enhancer) and is capable of functioning as a vector in the sense understood by those of ordinary skill in the art. Appropriate phage and viral vectors include, but are not limited to, lambda (X) bacteriophage, EMBL bacteriophage, simian virus 40, bovine papilloma virus, Epstein-Barr virus, adenovirus, herpes virus, vaccinia virus, Moloney murine leukemia virus, Harvey murine sarcoma virus, murine mammary tumor virus, lentivirus and Rous sarcoma virus.

Desirably, the vector is an expression vector or a recombinant expression vector. The term “recombinant expression vector” refers in context of the present invention to a nucleic acid construct that allows for the expression of an mRNA, protein or polypeptide in a suitable host cell. The recombinant expression vector of the invention can be any suitable recombinant expression vector, and can be used to transform or transfect any suitable host. Suitable vectors include those designed for propagation and expansion or for expression or both, such as plasmids and viruses. Examples of animal expression vectors include pEUK-CI, pMAM and pMAMneo. In an illustrated example, the recombinant expression vector is a viral vector, e.g., a retroviral vector. The recombinant expression vector comprises regulatory sequences, such as transcription and translation initiation and termination codons, which are specific to the type of host cell (e.g., bacterium, fungus, plant, or animal) into which the vector is to be introduced and in which the expression of the nucleic acid of the invention shall be performed. Furthermore, the vector of the invention may include one or more marker genes, which allow for selection of transformed or transfected hosts. The recombinant expression vector can comprise a native or normative promoter operably linked to the nucleotide sequence encoding the constructs of the invention, or to the nucleotide sequence which is complementary to or which hybridizes to the nucleotide sequence encoding the constructs of the invention. The selection of promoters include, e.g., strong, weak, inducible, tissue-specific and developmental-specific promoters. The promoter can be a non-viral promoter or a viral promoter. The inventive recombinant expression vectors can be designed for either transient expression, for stable expression, or for both. Also, the recombinant expression vectors can be made for constitutive expression or for inducible expression.

The invention will be further explained in the following making reference to either, several or all of these aspects. If reference is only made to one of these aspects, it is understood by the person skilled in the art, that this reference nevertheless includes references to all other aspects of the invention, if applicable.

In the fifth aspect, the invention is directed to a host cell comprising the antigen recognizing construct or T cell receptor according to the first or second aspect of the invention, or the nucleic acid sequence according to the third aspect of the invention, or the vector according to the fourth aspect of the invention.

Specifically, the host cell of the invention comprises a nucleic acid, or a vector as described herein above. The host cell can be an eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. For purposes of producing a recombinant TCR, polypeptide, or protein, the host cell is e.g. a mammalian cell. In another illustrated example, the host cell is a human cell. While the host cell can be of any cell type, it can originate from any type of tissue, and can be of any developmental stage, the host cell may be a peripheral blood leukocyte (PBL) or a peripheral blood mononuclear cell (PBMC). In another illustrated example, the host cell is a T cell. The T cell can be any T cell, such as a cultured T cell, e.g., a primary T cell, or a T cell from a cultured T cell line, e.g., Jurkat, SupTI, etc., or a T cell obtained from a mammal, specifically a T cell or T cell precurser from a human patient. If obtained from a mammal, the T cell can be obtained from numerous sources, including, but not limited to, blood, bone marrow, lymph node, the thymus, or other tissues or fluids. T cells can also be enriched for or purified. In another illustrated example, the T cell is a human T cell. In another illustrated example, the T cell is a T cell isolated from a human. The T cell can be any type of T cell and can be of any developmental stage, including, but not limited to, CD4 positive and/or CD8 positive, CD4 positive helper T cells, e.g., Th1 and Th2 cells, CD8 positive T cells (e.g., cytotoxic T cells), tumor infiltrating lymphocytes (TILs), memory T cells, naïve T cells, and the like. In another illustrated example, the T cell is a CD8 positive T cell or a CD4 positive T cell.

In an illustrative example, the host cell of the invention is a lymphocyte, specifically a T lymphocyte, such as a CD4 or CD8 positive T-cell. The host cell furthermore may be specifically a tumor reactive T cell specific for mutated KRAS expressing tumor cells.

In the sixth aspect, the invention is directed to the antigen recognizing construct or T cell receptor according to the first or second aspect of the invention, or the nucleic acid sequence according to the third aspect of the invention, or the vector according to the fourth aspect of the invention, or the host cell according to the fifth aspect of the invention, for use in medicine.

In the seventh aspect, the invention is directed to the antigen recognizing construct or T cell receptor according to the first or second aspect of the invention, or the nucleic acid sequence according to the third aspect of the invention, or the vector according to the fourth aspect of the invention, or the host cell according to the fifth aspect of the invention, for use in the prevention and/or treatment of a disease.

The terms “treat” and “prevent” as well as words stemming therefrom, like “treatment” or “prevention”, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the cancer being treated or prevented. For example, treatment or prevention can include promoting the regression of a tumor. Also, for purposes herein, “prevention” can encompass delaying the onset of the cancer, or a symptom or condition thereof.

According to the sixth or the seventh aspect of the invention, the disease may be a malignant or benign tumor disease.

The term “tumor” or “tumor disease” in the context of the present invention denotes a disease selected from e.g. melanomas, hepatocellular carcinomas, intra- and extrahepatic cholangiocellular carcinomas, squamous cell carcinomas, adenocarcinomas as well as undifferentiated carcinomas of the head, neck, lung or esophagus, colorectal carcinomas, chondrosarcomas, osteosarcomas, medulloblastomas, neuroblastomas, non-squamous cell carcinomas of the head or neck, ovarian tumors, lymphomas, acute and chronic lymphocytic leukemias, acute and chronic myeloid leukemia, bladder carcinomas, prostate carcinomas, pancreatic adenocarcinomas, mammary carcinomas and gastric carcinomas. In illustrated examples, diseases to be treated by the products and/or methods of the invention include melanoma, non-small-cell lung cancer, pancreatic adenocarcinoma and cholangiocellular carcinoma.

In the sixth or the seventh aspect of the invention, the disease may be a tumor that expresses mutated KRAS. Specifically, the disease may be an advanced-stage tumor that expresses mutated KRAS.

With regard to the sixth or the seventh aspect of the invention, the disease may be selected from the group consisting of Hodgkin's lymphoma, non-Hodgkin's lymphoma, acute myeloid leukemia, pancreatic cancer, colorectal cancer, endometrial cancer, biliary tract cancer, liver cancer, myeloma, prostate cancer, stomach cancer, kidney cancer, bone cancer, soft tissue cancer, head and neck cancer, glioblastoma multiforme, astrocytomas, melanoma, lung cancer, esophageal cancer, gastric cancer, breast cancer, ovarian cancer, mesothelioma cancer, bladder cancer, anal cancer, chondrosarcoma cancer, osteosarcoma cancer, sarcoma cancer, adenoma cancer, primitive neuroectodermal cancer (primitive neuroectodermal tumor (PNET)), and combinations thereof.

The lung cancer may be, but is not limited to, squamous cell carcinoma of the lung, non-small cell lung cancer and small cell lung cancer. In other illustrative examples, the breast cancer may be, but is not limited to, ductal breast cancer, tubular breast cancer, medullary breast cancer and combinations thereof. In yet another illustrative example, the gastric cancer may be gastric adenocarcinoma cancer. Turning to sarcoma cancer, the sarcoma cancer may be, but is no limited to, chondrosarcoma cancer, osteosarcoma cancer and combinations thereof. The adenoma cancer may include, but is also not limited to, gastric adenocarcinoma, pancreatic adenocarcinoma and combinations thereof.

The eighth aspect of the invention is directed to a use of the antigen recognizing construct or the T cell receptor according to the first or the second aspect of the invention, or the nucleic acid sequence according to the third aspect of the invention, or the vector according to the fourth aspect of the invention, or the host cell according to the fifth aspect of the invention, for the manufacture of a medicament for treating a disease. The disease may be a malignant or benign tumor disease, but is not limited thereto. In yet another illustrative example, the disease may be a tumor that expresses mutated KRAS. Without being limited to it, the disease is an advanced-stage tumor that expresses mutated KRAS.

With regard to the eighth aspect of the invention, the disease may be selected from the group consisting of Hodgkin's lymphoma, non-Hodgkin's lymphoma, acute myeloid leukemia, pancreatic cancer, colorectal cancer, endometrial cancer, biliary tract cancer, liver cancer, myeloma, prostate cancer, stomach cancer, kidney cancer, bone cancer, soft tissue cancer, head and neck cancer, glioblastoma multiforme, astrocytomas, melanoma, lung cancer, esophageal cancer, gastric cancer, breast cancer, ovarian cancer, mesothelioma cancer, bladder cancer, anal cancer, chondrosarcoma cancer, osteosarcoma cancer, sarcoma cancer, adenoma cancer, primitive neuroectodermal cancer (primitive neuroectodermal tumor (PNET)), and combinations thereof. The lung cancer may be, but is not limited to, squamous cell carcinoma of the lung, non-small cell lung cancer and small cell lung cancer. In other illustrative examples, the breast cancer may be, but is not limited to, ductal breast cancer, tubular breast cancer, medullary breast cancer and combinations thereof. In yet another illustrative example, the gastric cancer may be gastric adenocarcinoma cancer. Turning to sarcoma cancer, the sarcoma cancer may be, but is no limited to, chondrosarcoma cancer, osteosarcoma cancer and combinations thereof. The adenoma cancer may include, but is also not limited to, gastric adenocarcinoma, pancreatic adenocarcinoma and combinations thereof.

The ninth aspect of the invention is directed to a method of treating a disease, comprising the step of administering a therapeutically effective amount of the antigen recognizing construct or the T cell receptor according to the first or the second aspect of the invention, or the nucleic acid sequence according to the third aspect of the invention, or the vector according to the fourth aspect of the invention, or the host cell according to the fifth aspect of the invention. The disease may be a malignant or benign tumor disease, but is not limited thereto. In yet another illustrative example, the disease may be a tumor that expresses mutated KRAS. Without being limited to it, the disease is an advanced-stage tumor that expresses mutated KRAS.

With regard to the ninth aspect of the invention, the disease may be selected from the group consisting of Hodgkin's lymphoma, non-Hodgkin's lymphoma, acute myeloid leukemia, pancreatic cancer, colorectal cancer, endometrial cancer, biliary tract cancer, liver cancer, myeloma, prostate cancer, stomach cancer, kidney cancer, bone cancer, soft tissue cancer, head and neck cancer, glioblastoma multiforme, astrocytomas, melanoma, lung cancer, esophageal cancer, gastric cancer, breast cancer, ovarian cancer, mesothelioma cancer, bladder cancer, anal cancer, chondrosarcoma cancer, osteosarcoma cancer, sarcoma cancer, adenoma cancer, primitive neuroectodermal cancer (primitive neuroectodermal tumor (PNET)), and combinations thereof. The lung cancer may be, but is not limited to, squamous cell carcinoma of the lung, non-small cell lung cancer and small cell lung cancer. In other illustrative examples, the breast cancer may be, but is not limited to, ductal breast cancer, tubular breast cancer, medullary breast cancer and combinations thereof. In yet another illustrative example, the gastric cancer may be gastric adenocarcinoma cancer. Turning to sarcoma cancer, the sarcoma cancer may be, but is no limited to, chondrosarcoma cancer, osteosarcoma cancer and combinations thereof. The adenoma cancer may include, but is also not limited to, gastric adenocarcinoma, pancreatic adenocarcinoma and combinations thereof.

Addressing now in more detail the treatment of the disease, e.g. the tumor, the method of treatment can apply any suitable cancer treatment, for example, a treatment with a small molecule chemotherapeutic drug or immunotherapy. An immunotherapy treatment may comprise administering to the patient a therapeutically effective amount of an adoptive cell therapy agent or an agent specifically binding mutated KRAS.

Examples of cell therapy agents may be genetically modified cells of the immune systems such as T cells or natural killer (NK) cells. Such genetically modified cells include chimeric antigen receptor T-cells (CAR T-cells, see Jakobsen & Gjerstorff “CAR T-Cell Cancer Therapy Targeting Surface Cancer/Testis Antigens”, Front. Immunol. 2 Sep. 2020, Article 01568, doi: 10.3389/fimmu.2020.01568) or genetically modified T-cells that express a T cell receptor that specifically binds mutated KRAS. The genetically modified cells may be autologous cells derived from the patient to be treated but also allogeneic cells, i.e., cells that are obtained not from the patient of interest, but cells that have been derived from a “universal” donor cell. This “universal” donor cell may be derived from naturally occurring cells, such T cells or NK cells from a human donor (cf., in this respect, for example, see the review article of Perez et al., Off-the-Shelf Allogeneic T Cell Therapies for Cancer: Opportunities and Challenges Using Naturally Occurring “Universal” Donor T Cells”, Front. Immunol., 11 Nov. 2020, Article 583716, https://doi.org/10.3389/fimmu.2020.583716). It is however also possible to derive such “universal” donor cells from induced pluripotent stem cells (iPSC), cf. the review article of Flahou et al., “Fit-For-All iPSC-Derived Cell Therapies and Their Evaluation in Humanized Mice With NK Cell Immunity” Front. Immunol., 2 Apr. 2021, Article 662360, https://doi.org/10.3389/fimmu.2021.662360). In case genetically modified T cells are used as a cell therapy agent, the T cells may be of any suitable phenotype, for example, but not limited to, CD8+ T cells, CD4+ T cells or a combination thereof. Regardless of whether autologous (patient derived) T cells or allogeneic T cells are used, the T cells may express a recombinant T cell receptor (TCR) that specifically binds mutated KRAS.

When cellular product/agents such as genetically modified T cells are used for the method of treatment of the invention, such cells can be used in any suitable dosage (therapeutically effective amount). The dosage of the T cells administered to the patient, defined as the total number of T cells, may be from about 0.5×10⁷ T cells to about 1×10¹⁰ T cells. Exemplary dosages of the T cells administered to the patient, defined as the total number of T cells, may be about 0.75×10⁸ T cells, about 1×10⁸ cells T cells, about 1×10⁹ T cells, about 3×10⁹ T cells, about 4×10⁹ T cells, about 5×10⁹ T cells, about 6×10⁹ T cells, about 7×10⁹ T cells, about 8×10⁹ T cells, or about 9×10⁹ T cells. The dosage of the T cells administered to the patient, defined as the total number of T cells, may thus be in the range of about 1×10⁹ cells to about 9×10⁹ cells or in the rage of about 3×10⁹ cells to about 9×10⁹ cells. It is noted here that as used herein with respect to the dosage/number of cells used for administration, the term “about” means to include a deviation from the respective value of up to 1%, of up to 2%, of up to 3%, of up to 4%, of up to 5%, or up to and including 10% of the given value. This means, for example, a dosage of “about 1×10⁹ T cells” may include a total number of cells ranging from 1×10⁹%±10%, i.e. from 0.9×10⁹ to 1.1×10⁹ of T cells expressing a mutated KRAS binding TCR.

In accordance with the above disclosure, the invention also provides a pharmaceutical composition comprising the antigen recognizing construct or the T cell receptor according to the first or the second aspect of the invention, or the nucleic acid sequence according to the third aspect of the invention, or the vector according to the fourth aspect of the invention, or the host cell according to the fifth aspect of the invention.

This pharmaceutical composition may be in any suitable form, (depending upon the desired method of administering it to a patient). It may be provided in unit dosage form, will generally be provided in a sealed container and may be provided as part of a kit. Such a kit would normally (although not necessarily) include instructions for use. It may include a plurality of said unit dosage forms. Suitable compositions and methods of administration are known to those skilled in the art, for example, see Johnson et al., Blood. 2009 Jul. 16; 114(3): 535-46, with reference to clinical trial numbers NCI-07-C-0175 and NCI-07-C-0174. Cells in accordance may be supplied as part of a sterile, pharmaceutical composition, which will normally include a pharmaceutically acceptable carrier. For example, T cells transfected with TCRs of the invention may be provided in pharmaceutical composition together with a pharmaceutically acceptable carrier. The pharmaceutically acceptable carrier may be a cream, emulsion, gel, liposome, nanoparticle or ointment.

The pharmaceutical composition may be adapted for administration by any appropriate route such as a parenteral (including subcutaneous, intramuscular, or intravenous), enteral (including oral or rectal), inhalation or intranasal routes. Such compositions may be prepared by any method known in the art of pharmacy, for example, by mixing the active ingredient with the carrier(s) or excipient(s) under sterile conditions.

In illustrative examples of the pharmaceutical composition of the invention, the total number of T cells comprised in the composition may be about 0.75×10⁸ T cells, about 1×10⁸ cells, about 1×10⁹ T cells, about 3×10⁹ T cells, about 4×10⁹ T cells, about 5×10⁹ T cells, about 6×10⁹ T cells, about 7×10⁹ T cells, about 8×10⁹ T cells or about 9×10⁹ T cells.

The pharmaceutical composition may further comprise one or more pharmaceutically acceptable carrier. Any pharmaceutically acceptable carrier can be used, as long as the carrier does not impact the viability of the T cells to be administered and as long as the carrier is suitable for the chosen route of administration of the pharmaceutical composition. The pharmaceutical acceptable carrier may be a physiological saline solution, optionally with components such as human serum albumin that can improve the viability of the T cells that express the mutant KRAS binding TCR. It is also possible that the mutant KRAS binding T cells are stored, after their manufacture, in frozen form, for example, at a temperature of between −20° C. and −80° C. In this case, the pharmaceutical composition may contain cryo-protectants that have been added to protect the cells from being damaged by the freezing process. Examples of cryoprotectants that may be used here for the freezing of the pharmaceutical composition containing transduced T cells include glycerol, DMSO. These cryoprotectant can be used together with crystalloid solutions such as commercially available HypoThermosol® or PlasmaLyte-A solution, which are both approved for infusion and are available in pharmaceutical grade. Other possible media that can be used as carrier in the pharmaceutical composition are media of the “CryoStor family”, commercially available animal protein-free defined cryopreservation media from Biolife Solutions such as CyroStor2 (CS2, an optimized freeze media pre-formulated with 2% DMSO), CyroStor5 (CS5, an optimized freeze media pre-formulated with 5% DMSO), or CyroStor10 (CS10, an optimized freeze media pre-formulated with 10% DMSO).

Dealing now with the kit of the invention, such a kit is a kit for use in medicine comprising the antigen recognizing construct or the T cell receptor according to the first or the second aspect of the invention, or the nucleic acid sequence according to the third aspect of the invention, or the vector according to the fourth aspect of the invention, or the host cell according to the fifth aspect of the invention. Without being limited to it, the kit may be a diagnostic kit for selecting a patient for treatment of a tumor, wherein cells of the tumor express mutated KRAS.

The invention will be further illustrated by the following non-limiting Experimental Examples.

Sequences, as used herein, are depicted in below Table 1 and Table 2.

TABLE 1
Sequences as used herein.

| SEQ | | |
|---|---|---|
| ID NO: | Name | Sequence |
| 1 | epitope | VVGAVGVGK |
| 2 | alpha chain CDR 1 of | NSAFQY |
| | TCR 3-14 | |
| 3 | alpha chain CDR2 of | TYSSGN |
| | TCR 3-14 | |
| 4 | alpha chain CDR3 of | CAMSAGQAGTALIF |
| | TCR 3-14 | |
| 5 | beta chain CDR1 of | SGHNS |
| | TCR 3-14 | |
| 6 | beta chain CDR2 of | FNNNVP |
| | TCR 3-14 | |
| 7 | beta chain CDR3 of | CASSPWTPDTQYF |
| | TCR 3-14 | |
| 8 | alpha chain CDR1 of | TSINN |
| | TCR 4-13 | |
| 9 | alpha chain CDR2 of | IRSNERE |
| | TCR 4-13 | |
| 10 | alpha chain CDR3 of | CATDRNSGNTPLVF |
| | TCR 4-13 | |
| 11 | beta chain CDR1 of | SGHAT |
| | TCR 4-13 | |
| 12 | beta chain CDR2 of | FQNNGV |
| | TCR 4-13 | |
| 13 | beta chain CDR3 of | CASSLDSFYEQYF |
| | TCR 4-13 | |
| 14 | alpha chain CDR1 of | NSASDY |
| | TCR 3-13 | |
| 15 | alpha chain CDR2 of | IRSNMDK |
| | TCR 3-13 | |
| 16 | alpha chain CDR3 of | CAENNFGNEKLTF |
| | TCR 3-13 | |
| 17 | beta chain CDR1 of | DFQATT |
| | TCR 3-13 | |
| 18 | beta chain CDR2 of | SNEGSKA |
| | TCR 3-13 | |
| 19 | beta chain CDR3 of | CSASTSGRWYNEQFF |
| | TCR 3-13 | |
| 20 | alpha chain CDR1 of | NSAFQY |
| | TCR 3-7 | |
| 21 | alpha chain CDR2 of | TYSSGN |
| | TCR 3-7 | |
| 22 | alpha chain CDR3 of | CAMSVGTSGSRLTF |
| | TCR 3-7 | |
| 23 | beta chain CDR1 of | SGHNS |
| | TCR 3-7 | |
| 24 | beta chain CDR2 of | FNNNVP |
| | TCR 3-7 | |
| 25 | beta chain CDR3 of | CASSSWSPDTQYF |
| | TCR 3-7 | |
| 26 | alpha chain CDR1 of | DSASNY |
| | TCR 3-10 | |
| 27 | alpha chain CDR2 of | IRSNVGE |
| | TCR 3-10 | |
| 28 | alpha chain CDR3 of | CAATPISNFGNEKLTF |
| | TCR 3-10 | |
| 29 | beta chain CDR1 of | MNHEY |
| | TCR 3-10 | |
| 30 | beta chain CDR2 of | SMNVEV |
| | TCR 3-10 | |
| 31 | beta chain CDR3 of | CASSLSIVNEQFF |
| | TCR 3-10 | |

TABLE 2
Further sequences as used herein.

| TCR | alpha | beta |
|---|---|---|
| TCR | METLLGVSLVILWLQLARVNSQQ | MGTRLLCWAALCLLGAELTEAGVAQSP |
| 4-13 | GEEDPQALSIQEGENATMNCSYK | RYKIIEKRQSVAFWCNPISGHATLYWY |
| | TSINNLQWYRQNSGRGLVHLILIR | QQILGQGPKLLIQFQNNGVVDDSQLPK |
| | SNEREKHSGRLRVTLDTSKKSSS | DRFSAERLKGVDSTLKIQPAKLEDSAVY |
| | LLITASRAADTASYFCATDRNSG | LCASSLDSFYEQYFGPGTRLTVTEDLK |
| | NTPLVFGKGTRLSVIANIQNPDPA | NVFPPEVAVFEPSKAEIAHTQKATLVC |
| | VYQLRDSKSSDKSVCLFTDFDS | LATGFYPDHVELSWWVNGKEVHSGVS |
| | QTNVSQSKDSDVYITDKTVLDMR | TDPQPLKEQPALNDSRYCLSSRLRVS |
| | SMDFKSNSAVAWSNKSDFACA | ATFWQNPRNHFRCQVQFYGLSENDE |
| | NAFNNSIIPEDTFFPSSDVPCDVK | WTQDRAKPVTQIVSAEAWGRADCGIT |
| | LVEKSFETDTNLNFQNLSVIGFRI | SASYHQGVLSATILYEILLGKATLYAVL |
| | LLLKVAGFNLLMTLRLWSS | VSALVLMAMVKRKDSRG |
| | (SEQ ID NO: 32) | (SEQ ID NO: 33) |
| TCR | MAGIRALFMYLWLQLDWVSRGE | MLLLLLLLGPGSGLGAVVSQHPSWVIC |
| 3-13 | SVGLHLPTLSVQEGDNSIINCAYS | KSGTSVKIECRSLDFQATTMFWYRQFP |
| | NSASDYFIWYKQESGKGPQFIIDI | KQSLMLMATSNEGSKATYEQGVEKDK |
| | RSNMDKRQGQRVTVLLNKTVKH | FLINHASLTLSTLTVTSAHPEDSSFYICS |
| | LSLQIAATQPGDSAVYFCAENNF | ASTSGRWYNEQFFGPGTRLTVLEDLK |
| | GNEKLTFGTGTRLTIIPNIQNPDP | NVFPPEVAVFEPSKAEIAHTQKATLVC |
| | AVYQLRDSKSSDKSVCLFTDFD | LATGFYPDHVELSWWVNGKEVHSGVS |
| | SQTNVSQSKDSDVYITDKTVLDM | TDPQPLKEQPALNDSRYCLSSRLRVS |
| | RSMDFKSNSAVAWSNKSDFAC | ATFWQNPRNHFRCQVQFYGLSENDE |
| | ANAFNNSIIPEDTFFPSSDVPCDV | WTQDRAKPVTQIVSAEAWGRADCGIT |
| | KLVEKSFETDTNLNFQNLSVIGF | SASYHQGVLSATILYEILLGKATLYAVL |
| | RILLLKVAGFNLLMTLRLWSS | VSALVLMAMVKRKDSRG |
| | (SEQ ID NO: 34) | (SEQ ID NO: 35) |
| TCR | MMKSLRVLLVILWLQLSWVWSQ | MDSWTFCCVSLCILVAKHTDAGVIQSP |
| 3-14 | QKEVEQDPGPLSVPEGAIVSLNC | RHEVTEMGQEVTLRCKPISGHNSLFWY |
| | TYSNSAFQYFMWYRQYSRKGPE | RQTMMRGLELLIYFNNNVPIDDSGMPE |
| | LLMYTYSSGNKEDGRFTAQVDK | DRFSAKMPNASFSTLKIQPSEPRDSAV |
| | SSKYISLFIRDSQPSDSATYLCAM | YFCASSPWTPDTQYFGPGTRLTVLEDL |
| | SAGQAGTALIFGKGTTLSVSSNIQ | KNVFPPEVAVFEPSKAEIAHTQKATLV |
| | NPDPAVYQLRDSKSSDKSVCLF | CLATGFYPDHVELSWWVNGKEVHSG |
| | TDFDSQTNVSQSKDSDVYITDKT | VSTDPQPLKEQPALNDSRYCLSSRLR |
| | VLDMRSMDFKSNSAVAWSNKS | VSATFWQNPRNHFRCQVQFYGLSEND |
| | DFACANAFNNSIIPEDTFFPSSDV | EWTQDRAKPVTQIVSAEAWGRADCGI |
| | PCDVKLVEKSFETDTNLNFQNLS | TSASYHQGVLSATILYEILLGKATLYAV |
| | VIGFRILLLKVAGFNLLMTLRLWS | LVSALVLMAMVKRKDSRG |
| | S | (SEQ ID NO: 37) |
| | (SEQ ID NO: 36) | |
| TCR | MMKSLRVLLVILWLQLSWVWSQ | MDSWTFCCVSLCILVAKHTDAGVIQSP |
| 3-7 | QKEVEQDPGPLSVPEGAIVSLNC | RHEVTEMGQEVTLRCKPISGHNSLFWY |
| | TYSNSAFQYFMWYRQYSRKGPE | RQTMMRGLELLIYFNNNVPIDDSGMPE |
| | LLMYTYSSGNKEDGRFTAQVDK | DRFSAKMPNASFSTLKIQPSEPRDSAV |
| | SSKYISLFIRDSQPSDSATYLCAM | YFCASSSWSPDTQYFGPGTRLTVLEDL |
| | SVGTSGSRLTFGEGTQLTVNPDI | KNVFPPEVAVFEPSKAEIAHTQKATLV |
| | QNPDPAVYQLRDSKSSDKSVCL | CLATGFYPDHVELSWWVNGKEVHSG |
| | FTDFDSQTNVSQSKDSDVYITDK | VSTDPQPLKEQPALNDSRYCLSSRLR |
| | TVLDMRSMDFKSNSAVAWSNKS | VSATFWQNPRNHFRCQVQFYGLSEND |
| | DFACANAFNNSIIPEDTFFPSSDV | EWTQDRAKPVTQIVSAEAWGRADCGI |
| | PCDVKLVEKSFETDTNLNFQNLS | TSASYHQGVLSATILYEILLGKATLYAV |
| | VIGFRILLLKVAGFNLLMTLRLWS | LVSALVLMAMVKRKDSRG |
| | S | (SEQ ID NO: 39) |
| | (SEQ ID NO: 38) | |
| TCR | MTSIRAVFIFLWLQLDLVNGENVE | MGPQLLGYVVLCLLGAGPLEAQVTQNP |
| 3-10 | QHPSTLSVQEGDSAVIKCTYSDS | RYLITVTGKKLTVTCSQNMNHEYMSWY |
| | ASNYFPWYKQELGKGPQLIIDIRS | RQDPGLGLRQIYYSMNVEVTDKGDVP |
| | NVGEKKDQRIAVTLNKTAKHFSL | EGYKVSRKEKRNFPLILESPSPNQTSLY |
| | HITETQPEDSAVYFCAATPISNFG | FCASSLSIVNEQFFGPGTRLTVLEDLKN |
| | NEKLTFGTGTRLTIIPNIQNPDPA | VFPPEVAVFEPSKAEIAHTQKATLVCL |
| | VYQLRDSKSSDKSVCLFTDFDS | ATGFYPDHVELSWWVNGKEVHSGVST |
| | QTNVSQSKDSDVYITDKTVLDMR | DPQPLKEQPALNDSRYCLSSRLRVSA |
| | SMDFKSNSAVAWSNKSDFACA | TFWQNPRNHFRCQVQFYGLSENDEW |
| | NAFNNSIIPEDTFFPSSDVPCDVK | TQDRAKPVTQIVSAEAWGRADCGITS |
| | LVEKSFETDTNLNFQNLSVIGFRI | ASYHQGVLSATILYEILLGKATLYAVLV |
| | LLLKVAGFNLLMTLRLWSS | SALVLMAMVKRKDSRG |
| | (SEQ ID NO: 40) | (SEQ ID NO: 41) |

Variable regions are underlined, constant regions are in bold. The term “variable region” may also be called “variable domain” as used herein, both terms may be used synonymously. The term “constant region” may also be called “constant domain” as used herein, both terms may be used synonymously.

These sequences of SEQ ID NOs: 32 to 41 are also depicted in FIG. 5 given herein.

In the constant domains of the TCRs as described herein and in Table 2, “minimal murinization” may be carried out, e.g. by replacing four positions of the human α chain and five positions of the human β chain by the respective murine amino acids, for example, by murine residues Ser 90, Asp 91, Val 92 and Pro 93 in the human α chain and murine residues Lys18, Ala22, Ile133, Ala136, and His139 in the human β chain.

Alternatively, the fully humanized sequence may be used in the constant domains as described herein and as depicted in Table 2. Such a humanized sequence of the α chain is e.g. deposited under accession number UniProtKB—P01848 (TRAC_HUMAN) and a humanized sequence of the R chain is e.g. deposited under accession number UniProtKB—A0A51B9 (TRBC2_HUMAN).

### EXPERIMENTAL EXAMPLES

### Example 1

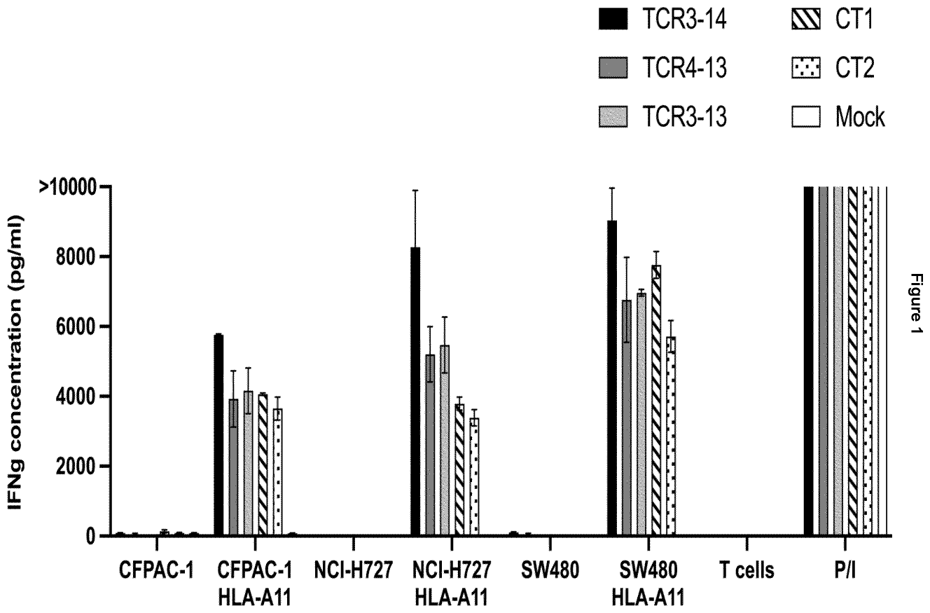

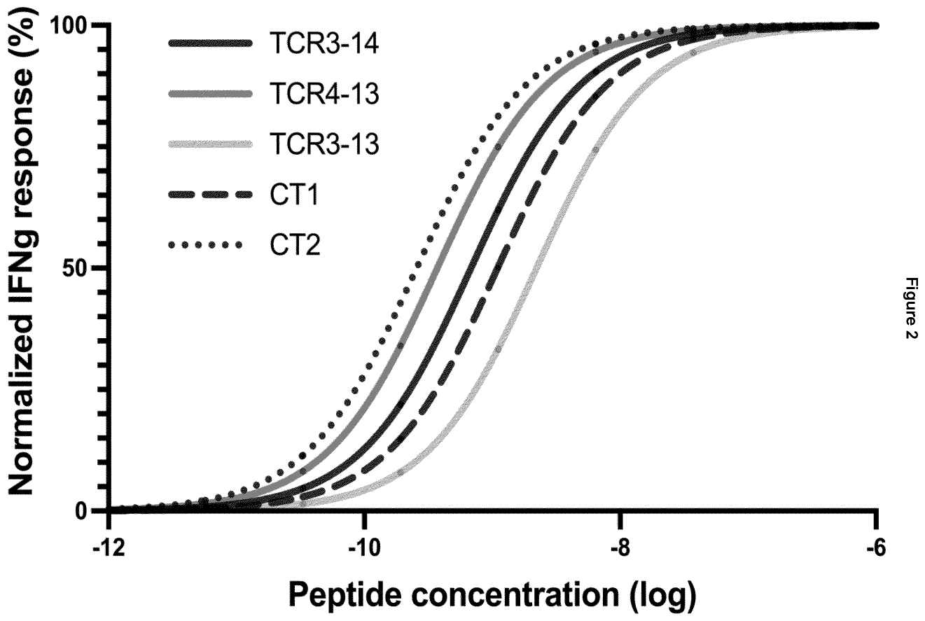

TCR- or mock-transduced peripheral blood leukocytes (PBLs) from a human donor were co-cultured with mutant KRAS (KRAS^{G12V}) expressing cell lines (intact or transduced with HLA-A*11:01) or alone (“T cells”). PMA and ionomycin stimulation (“P/I”) was used as a positive control of T cell activation. IFNγ secretion in the culture supernatants upon T cell activation was measured by a sandwich ELISA. The results of this Example are shown in FIG. 1 included herein.

The inventors of the invention have always additionally created the data for 2 control TCRs, named CT1 and CT2 herein, which were derived from a human donor and from an immunized mouse. Those controls are according to Cafri et al. (“Memory T cells targeting oncogenic mutations detected in peripheral blood of epithelial cancer patients”. Nat. Commun. 10, 449, 2019) and from Wang et al. (“Identification of T-cell Receptors Targeting KRAS-Mutated Human Tumors”. Cancer Immunol. Res. 2016 March; 4(3): 204-14. doi: 10.1158/2326-6066.CIR-15-0188) (see also WO 2016/085904 A1 and WO 2019/112941 A1).

### Example 2

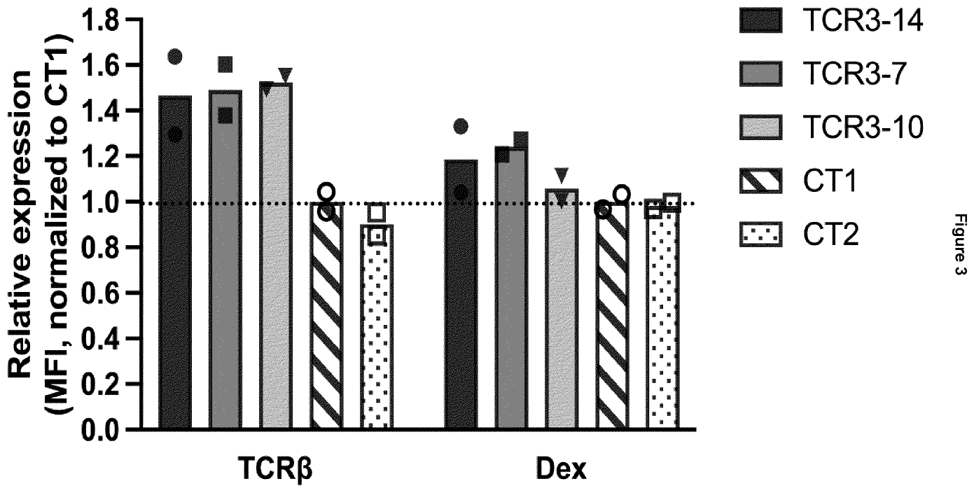

TCR-transduced PBLs from a human donor were co-cultured with HLA-A*11:01 expressing cells loaded with the recognized peptide (VVGAVGVGK, SEQ ID NO: 1) at descending concentrations (10⁻⁶ M to 10⁻¹² M). IFNγ secretion in the culture supernatants upon T cell activation was measured by a sandwich ELISA. Normalized IFNγ secretion curves are shown in FIG. 2 included herein.

### Example 3

Expression of TCRs on the surface of TCR-transduced CD8+ T cells from two donors was analyzed by flow cytometry. The results thereof are shown herein in FIG. 3. TCR expression was measured by staining the cells with anti-mouse TCR β-chain or with the HLA-A*11:01-VVGAVGVGK dextramer (“Dex”). The TCR expression was represented as mean fluorescence intensity (MFI) normalized to CT1 TCR.

### Example 4

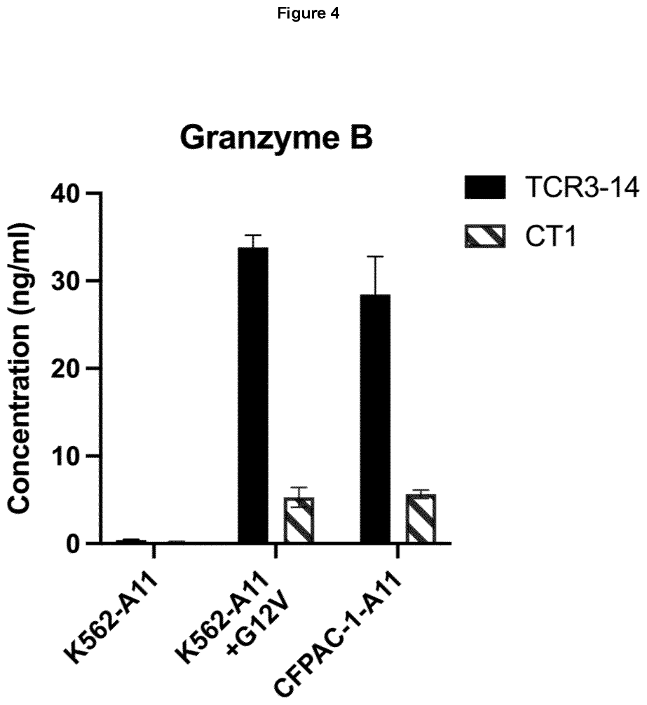

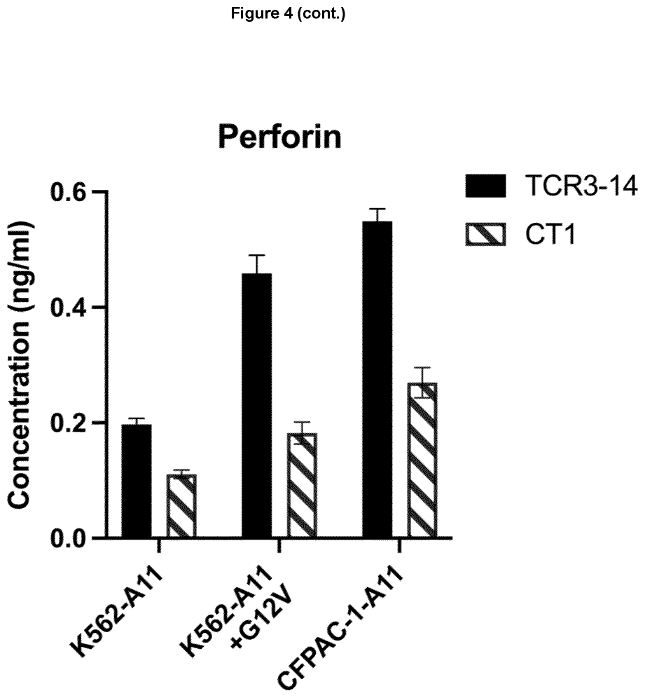

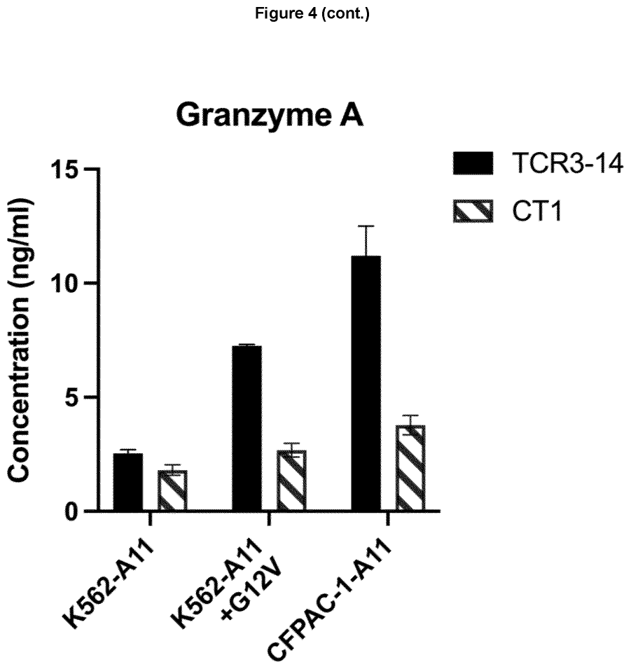

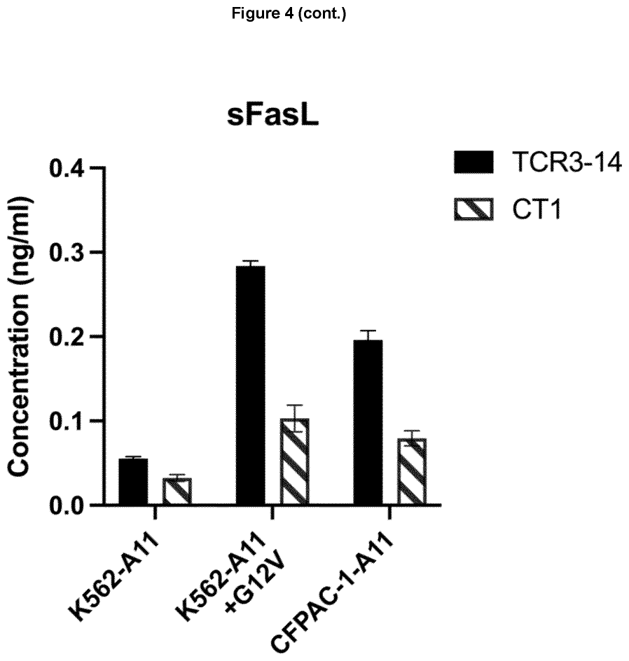

TCR-transduced human PBLs were co-cultured with HLA-A*11:01-expressing cell lines. The results thereof are shown herein in FIG. 4. CFPAC-1 cells that express KRAS^{G12V} (“CFPAC-1-A11”) or K-562 cells were loaded with the recognized peptide (VVGAVGVGK; 10⁻⁶ M) (“K562-A11+G12V”). Non-loaded K-562 cells served as a negative control (“K562-A11”). Protein secretion in the culture supernatants upon T cell activation was measured by LEGENDplex multi-analyte flow assay kit (Biolegend) using the human CD8/NK panel following the manufacturer's instructions.

### Materials and Methods

### Animal Immunizations

To generate CD8+ T cell responses to KRAS^{G12V} epitopes, humanized TCR/HLA class I transgenic mice, which are a derivative from the ABabDII mice described in Li et al. (2010, Nature Medicine 16, 1029-1034), were used for immunizations with either a peptide or adenoviral vector.

For peptide immunizations, the mice were subcutaneously primed and boosted monthly with the KRAS^{G12V} peptide VVGAVGVGK supplemented with CpG1826 oligonucleotide and emulsified in incomplete Freund's adjuvant.

For adenoviral immunizations, the mice received intraperitoneal injections of a human type 5 (dE1/E3) adenoviral vector that expresses a KRAS^{G12V} minigene (which encodes amino acids 1-35) under control of the CMV promoter.

Specific T cell responses were monitored by re-stimulating peripheral blood with the KRAS^{G12V} peptide or the minigene-expressing cell lines in vitro one week after a boost.

### TCR Identification and Characterization

Splenocytes from responder mice were restimulated in vitro as described, and antigen-specific activated CD3+CD8+CD137+ T cells were sorted for cDNA library preparation and single-cell TCR sequencing using the 10× Genomics technology, following the manufacturer's protocols. Expanded TCR clonotypes were cloned into a retroviral vector and transduced into human peripheral blood lymphocytes.

The invention will be further illustrated by the following non-limiting items:

1. An antigen recognizing construct that binds the peptide VVGAVGVGK (SEQ ID NO: 1) with determinable affinity,
wherein the antigen recognizing construct comprises a complementary determining region 3 (CDR3) of the α-chain having at least 90% sequence identity to an amino acid sequence selected from the group consisting of SEQ ID NOs: 4, 10, 16, 22, and 28, and/or
wherein the antigen recognizing construct comprises a complementary determining region 3 (CDR3) of the β-chain having at least 90% sequence identity to an amino acid sequence selected from the group consisting of SEQ ID NOs: 7, 13, 19, 25, and 31.

2. The antigen recognizing construct of item 1, wherein the antigen recognizing construct is an antibody, or fragment thereof, or a T cell receptor (TCR), or fragment thereof.

3. The antigen recognizing construct of item 1 or item 2, wherein the antigen recognizing construct specifically binds the peptide VVGAVGVGK (SEQ ID NO: 1) with an EC₅₀ of less than about 1×10⁻⁶ M.

4. The antigen recognizing construct of item 3, wherein the EC₅₀ value is determined by the procedure as carried out in Example 2.

5. The antigen recognizing construct of any one of the preceding items, wherein the antigen recognizing construct binds the peptide being presented by a MHC I molecule.

6. The antigen recognizing construct of item 5, wherein the MHC I molecule is an HLA-A molecule.

7. The antigen recognizing construct of item 6, wherein the HLA-A molecule is HLA-A*11:01.

8. The antigen recognizing construct of any one of the preceding items, wherein the antigen recognizing construct binds the peptide VVGAVGVGK (SEQ ID NO: 1) with an antigenic specificity of at least 80%.

9. The antigen recognizing construct of any one of the preceding items, wherein the antigen recognizing construct is a T cell receptor (TCR).

10. The T cell receptor of item 9, wherein the T cell receptor comprises a complementary determining region 1 (CDR1) of the α-chain having at least 90% sequence identity to an amino acid sequence selected from the group consisting of SEQ ID NOs: 2, 8, 14, 20, and 26, and/or wherein the T cell receptor comprises a complementary determining region 1 (CDR1) of the β-chain having at least 90% sequence identity to an amino acid sequence selected from the group consisting of SEQ ID NOs: 5, 11, 17, 23, and 29.

11. The T cell receptor of item 9 or 10, wherein the T cell receptor comprises a complementary determining region 2 (CDR2) of the α-chain having at least 90% sequence identity to an amino acid sequence selected from the group consisting of SEQ ID NOs: 3, 9, 15, 21, and 27, and/or wherein the T cell receptor comprises a complementary determining region 2 (CDR2) of the β-chain having at least 90% sequence identity to an amino acid sequence selected from the group consisting of SEQ ID NOs: 6, 12, 18, 24, and 30.

12. The T cell receptor of any one of items 9 to 11, wherein the T cell receptor comprises a complementary determining region 3 (CDR3) of the α-chain having at least 95% sequence identity to an amino acid sequence selected from the group consisting of SEQ ID NOs: 4, 10, 16, 22, and 28, and/or wherein the T cell receptor comprises a complementary determining region 3 (CDR3) of the β-chain having at least 95% sequence identity to an amino acid sequence selected from the group consisting of SEQ ID NOs: 7, 13, 19, 25, and 31.

13. The T cell receptor of any one of items 9 to 12, wherein the T cell receptor comprises a complementary determining region 1 (CDR1) of the α-chain having at least 95% sequence identity to an amino acid sequence selected from the group consisting of SEQ ID NOs: 2, 8, 14, 20, and 26, and/or wherein the T cell receptor comprises a complementary determining region 1 (CDR1) of the β-chain having at least 95% sequence identity to an amino acid sequence selected from the group consisting of SEQ ID NOs: 5, 11, 17, 23, and 29.

14. The T cell receptor of any one of items 9 to 13, wherein the T cell receptor comprises a complementary determining region 2 (CDR2) of the α-chain having at least 95% sequence identity to an amino acid sequence selected from the group consisting of SEQ ID NOs: 3, 9, 15, 21, and 27, and/or wherein the T cell receptor comprises a complementary determining region 2 (CDR2) of the β-chain having at least 95% sequence identity to an amino acid sequence selected from the group consisting of SEQ ID NOs: 6, 12, 18, 24, and 30.

15. The T cell receptor of any one of items 9 to 14, comprising:
(a) an α-chain CDR1 comprising or consisting of the amino acid sequence of SEQ ID NO: 2,
an α-chain CDR2 comprising or consisting of the amino acid sequence of SEQ ID NO: 3, and
an α-chain CDR3 comprising or consisting of the amino acid sequence of SEQ ID NO: 4; or
(b) an α-chain CDR1 comprising or consisting of the amino acid sequence of SEQ ID NO: 8,
an α-chain CDR2 comprising or consisting of the amino acid sequence of SEQ ID NO: 9, and
an α-chain CDR3 comprising or consisting of the amino acid sequence of SEQ ID NO: 10; or
(c) an α-chain CDR1 comprising or consisting of the amino acid sequence of SEQ ID NO: 14,
an α-chain CDR2 comprising or consisting of the amino acid sequence of SEQ ID NO: 15, and
an α-chain CDR3 comprising or consisting of the amino acid sequence of SEQ ID NO: 16; or
(d) an α-chain CDR1 comprising or consisting of the amino acid sequence of SEQ ID NO: 20,
an α-chain CDR2 comprising or consisting of the amino acid sequence of SEQ ID NO: 21, and
an α-chain CDR3 comprising or consisting of the amino acid sequence of SEQ ID NO: 22; or
(e) an α-chain CDR1 comprising or consisting of the amino acid sequence of SEQ ID NO: 26,
an α-chain CDR2 comprising or consisting of the amino acid sequence of SEQ ID NO: 27, and
an α-chain CDR3 comprising or consisting of the amino acid sequence of SEQ ID NO: 28.

16. The T cell receptor of any one of items 9 to 15, comprising:
(a) a β-chain CDR1 comprising or consisting of the amino acid sequence of SEQ ID NO: 5,
a β-chain CDR2 comprising or consisting of the amino acid sequence of SEQ ID NO: 6, and
a β-chain CDR3 comprising or consisting of the amino acid sequence of SEQ ID NO: 7; or
(b) a β-chain CDR1 comprising or consisting of the amino acid sequence of SEQ ID NO: 11,
a β-chain CDR2 comprising or consisting of the amino acid sequence of SEQ ID NO: 12, and
a β-chain CDR3 comprising or consisting of the amino acid sequence of SEQ ID NO: 13; or
(c) a β-chain CDR1 comprising or consisting of the amino acid sequence of SEQ ID NO: 17,
a β-chain CDR2 comprising or consisting of the amino acid sequence of SEQ ID NO: 18, and
a β-chain CDR3 comprising or consisting of the amino acid sequence of SEQ ID NO: 19; or
(d) a β-chain CDR1 comprising or consisting of the amino acid sequence of SEQ ID NO: 23,
a β-chain CDR2 comprising or consisting of the amino acid sequence of SEQ ID NO: 24, and
a β-chain CDR3 comprising or consisting of the amino acid sequence of SEQ ID NO: 25; or
(e) a β-chain CDR1 comprising or consisting of the amino acid sequence of SEQ ID NO: 29,
a β-chain CDR2 comprising or consisting of the amino acid sequence of SEQ ID NO: 30, and
a β-chain CDR3 comprising or consisting of the amino acid sequence of SEQ ID NO: 31.

17. The T cell receptor of any one of items 9 to 16, comprising:
(a) an α-chain CDR1 comprising or consisting of the amino acid sequence of SEQ ID NO: 2,
an α-chain CDR2 comprising or consisting of the amino acid sequence of SEQ ID NO: 3,
an α-chain CDR3 comprising or consisting of the amino acid sequence of SEQ ID NO: 4;
a β-chain CDR1 comprising or consisting of the amino acid sequence of SEQ ID NO: 5,
a β-chain CDR2 comprising or consisting of the amino acid sequence of SEQ ID NO: 6, and
a β-chain CDR3 comprising or consisting of the amino acid sequence of SEQ ID NO: 7; or
(b) an α-chain CDR1 comprising or consisting of the amino acid sequence of SEQ ID NO: 8,
an α-chain CDR2 comprising or consisting of the amino acid sequence of SEQ ID NO: 9,
an α-chain CDR3 comprising or consisting of the amino acid sequence of SEQ ID NO: 10;
a β-chain CDR1 comprising or consisting of the amino acid sequence of SEQ ID NO: 11,
a β-chain CDR2 comprising or consisting of the amino acid sequence of SEQ ID NO: 12, and
a β-chain CDR3 comprising or consisting of the amino acid sequence of SEQ ID NO: 13; or
(c) an α-chain CDR1 comprising or consisting of the amino acid sequence of SEQ ID NO: 14,
an α-chain CDR2 comprising or consisting of the amino acid sequence of SEQ ID NO: 15,
an α-chain CDR3 comprising or consisting of the amino acid sequence of SEQ ID NO: 16;
a β-chain CDR1 comprising or consisting of the amino acid sequence of SEQ ID NO: 17,
a β-chain CDR2 comprising or consisting of the amino acid sequence of SEQ ID NO: 18, and
a β-chain CDR3 comprising or consisting of the amino acid sequence of SEQ ID NO: 19; or
(d) an α-chain CDR1 comprising or consisting of the amino acid sequence of SEQ ID NO: 20,
an α-chain CDR2 comprising or consisting of the amino acid sequence of SEQ ID NO: 21,
an α-chain CDR3 comprising or consisting of the amino acid sequence of SEQ ID NO: 22;
a β-chain CDR1 comprising or consisting of the amino acid sequence of SEQ ID NO: 23,
a β-chain CDR2 comprising or consisting of the amino acid sequence of SEQ ID NO: 24, and
a β-chain CDR3 comprising or consisting of the amino acid sequence of SEQ ID NO: 25; or
(e) an α-chain CDR1 comprising or consisting of the amino acid sequence of SEQ ID NO: 26,
an α-chain CDR2 comprising or consisting of the amino acid sequence of SEQ ID NO: 27,
an α-chain CDR3 comprising or consisting of the amino acid sequence of SEQ ID NO: 28;
a β-chain CDR1 comprising or consisting of the amino acid sequence of SEQ ID NO: 29,
a β-chain CDR2 comprising or consisting of the amino acid sequence of SEQ ID NO: 30, and
a β-chain CDR3 comprising or consisting of the amino acid sequence of SEQ ID NO: 31.

18. A T cell receptor (TCR) having antigenic specificity for mutated KRAS, wherein the TCR comprises:
(a) an α-chain CDR1 comprising or consisting of the amino acid sequence of SEQ ID NO: 2,
an α-chain CDR2 comprising or consisting of the amino acid sequence of SEQ ID NO: 3, and
an α-chain CDR3 comprising or consisting of the amino acid sequence of SEQ ID NO: 4; or
(b) an α-chain CDR1 comprising or consisting of the amino acid sequence of SEQ ID NO: 8,
an α-chain CDR2 comprising or consisting of the amino acid sequence of SEQ ID NO: 9, and
an α-chain CDR3 comprising or consisting of the amino acid sequence of SEQ ID NO: 10; or
(c) an α-chain CDR1 comprising or consisting of the amino acid sequence of SEQ ID NO: 14,
an α-chain CDR2 comprising or consisting of the amino acid sequence of SEQ ID NO: 15, and
an α-chain CDR3 comprising or consisting of the amino acid sequence of SEQ ID NO: 16; or
(d) an α-chain CDR1 comprising or consisting of the amino acid sequence of SEQ ID NO: 20,
an α-chain CDR2 comprising or consisting of the amino acid sequence of SEQ ID NO: 21, and
an α-chain CDR3 comprising or consisting of the amino acid sequence of SEQ ID NO: 22; or
(e) an α-chain CDR1 comprising or consisting of the amino acid sequence of SEQ ID NO: 26,
an α-chain CDR2 comprising or consisting of the amino acid sequence of SEQ ID NO: 27, and
an α-chain CDR3 comprising or consisting of the amino acid sequence of SEQ ID NO: 28.

19. A T cell receptor (TCR) having antigenic specificity for mutated KRAS, wherein the TCR comprises:
(a) a β-chain CDR1 comprising or consisting of the amino acid sequence of SEQ ID NO: 5,
a β-chain CDR2 comprising or consisting of the amino acid sequence of SEQ ID NO: 6, and
a β-chain CDR3 comprising or consisting of the amino acid sequence of SEQ ID NO: 7; or
(b) a β-chain CDR1 comprising or consisting of the amino acid sequence of SEQ ID NO: 11,
a β-chain CDR2 comprising or consisting of the amino acid sequence of SEQ ID NO: 12, and
a β-chain CDR3 comprising or consisting of the amino acid sequence of SEQ ID NO: 13; or
(c) a β-chain CDR1 comprising or consisting of the amino acid sequence of SEQ ID NO: 17,
a β-chain CDR2 comprising or consisting of the amino acid sequence of SEQ ID NO: 18, and
a β-chain CDR3 comprising or consisting of the amino acid sequence of SEQ ID NO: 19; or
(d) a β-chain CDR1 comprising or consisting of the amino acid sequence of SEQ ID NO: 23,
a β-chain CDR2 comprising or consisting of the amino acid sequence of SEQ ID NO: 24, and
a β-chain CDR3 comprising or consisting of the amino acid sequence of SEQ ID NO: 25; or
(e) a β-chain CDR1 comprising or consisting of the amino acid sequence of SEQ ID NO: 29,
a β-chain CDR2 comprising or consisting of the amino acid sequence of SEQ ID NO: 30, and
a β-chain CDR3 comprising or consisting of the amino acid sequence of SEQ ID NO: 31.

20. A T cell receptor (TCR) having antigenic specificity for mutated KRAS, wherein the TCR comprises:
(a) an α-chain CDR1 comprising or consisting of the amino acid sequence of SEQ ID NO: 2,
an α-chain CDR2 comprising or consisting of the amino acid sequence of SEQ ID NO: 3,
an α-chain CDR3 comprising or consisting of the amino acid sequence of SEQ ID NO: 4; a β-chain CDR1 comprising or consisting of the amino acid sequence of SEQ ID NO: 5,
a β-chain CDR2 comprising or consisting of the amino acid sequence of SEQ ID NO: 6, and
a β-chain CDR3 comprising or consisting of the amino acid sequence of SEQ ID NO: 7; or
(b) an α-chain CDR1 comprising or consisting of the amino acid sequence of SEQ ID NO: 8,
an α-chain CDR2 comprising or consisting of the amino acid sequence of SEQ ID NO: 9,
an α-chain CDR3 comprising or consisting of the amino acid sequence of SEQ ID NO: 10;
a β-chain CDR1 comprising or consisting of the amino acid sequence of SEQ ID NO: 11,
a β-chain CDR2 comprising or consisting of the amino acid sequence of SEQ ID NO: 12, and
a β-chain CDR3 comprising or consisting of the amino acid sequence of SEQ ID NO: 13; or
(c) an α-chain CDR1 comprising or consisting of the amino acid sequence of SEQ ID NO: 14,
an α-chain CDR2 comprising or consisting of the amino acid sequence of SEQ ID NO: 15,
an α-chain CDR3 comprising or consisting of the amino acid sequence of SEQ ID NO: 16;
a β-chain CDR1 comprising or consisting of the amino acid sequence of SEQ ID NO: 17,
a β-chain CDR2 comprising or consisting of the amino acid sequence of SEQ ID NO: 18, and
a β-chain CDR3 comprising or consisting of the amino acid sequence of SEQ ID NO: 19; or
(d) an α-chain CDR1 comprising or consisting of the amino acid sequence of SEQ ID NO: 20,
an α-chain CDR2 comprising or consisting of the amino acid sequence of SEQ ID NO: 21,
an α-chain CDR3 comprising or consisting of the amino acid sequence of SEQ ID NO: 22;
a β-chain CDR1 comprising or consisting of the amino acid sequence of SEQ ID NO: 23,
a β-chain CDR2 comprising or consisting of the amino acid sequence of SEQ ID NO: 24, and
a β-chain CDR3 comprising or consisting of the amino acid sequence of SEQ ID NO: 25; or
(e) an α-chain CDR1 comprising or consisting of the amino acid sequence of SEQ ID NO: 26,
an α-chain CDR2 comprising or consisting of the amino acid sequence of SEQ ID NO: 27,
an α-chain CDR3 comprising or consisting of the amino acid sequence of SEQ ID NO: 28;
a β-chain CDR1 comprising or consisting of the amino acid sequence of SEQ ID NO: 29,
a β-chain CDR2 comprising or consisting of the amino acid sequence of SEQ ID NO: 30, and
a β-chain CDR3 comprising or consisting of the amino acid sequence of SEQ ID NO: 31.

21. A nucleic acid sequence encoding an antigen recognizing construct or T cell receptor as defined in any one of items 1 to 20.

22. A vector comprising a nucleic acid sequence as defined in item 21.

23. A host cell comprising the antigen recognizing construct or T cell receptor according to any one of items 1 to 20, or the nucleic acid sequence according to item 21, or the vector according to item 22.

24. The antigen recognizing construct or the T cell receptor according to any one of items 1 to 20, or the nucleic acid sequence according to item 21, or the vector according to item 22, or the host cell according to item 23, for use in medicine.

25. The antigen recognizing construct or the T cell receptor according to any one of items 1 to 20, or the nucleic acid sequence according to item 21, or the vector according to item 22, or the host cell according to item 23, for use in the prevention and/or treatment of a disease.

26. The antigen recognizing construct, or the T cell receptor, or the nucleic acid sequence, or the vector, or the host cell according to item 25, wherein the disease is a malignant or benign tumor disease.

27. The antigen recognizing construct, or the T cell receptor, or the nucleic acid sequence, or the vector, or the host cell according to item 25 or item 26, wherein the disease is a tumor that expresses mutated KRAS.

28. The antigen recognizing construct, or the T cell receptor, or the nucleic acid sequence, or the vector, or the host cell according to item 27, wherein the disease is an advanced-stage tumor that expresses mutated KRAS.

29. The antigen recognizing construct, or the T cell receptor, or the nucleic acid sequence, or the vector, or the host cell according to any one of items 25 to 28, wherein the disease is selected from the group consisting of Hodgkin's lymphoma, non-Hodgkin's lymphoma, acute myeloid leukemia, pancreatic cancer, colorectal cancer, endometrial cancer, biliary tract cancer, liver cancer, myeloma, prostate cancer, stomach cancer, kidney cancer, bone cancer, soft tissue cancer, head and neck cancer, glioblastoma multiforme, astrocytomas, melanoma, lung cancer, esophageal cancer, gastric cancer, breast cancer, ovarian cancer, mesothelioma cancer, bladder cancer, anal cancer, chondrosarcoma cancer, osteosarcoma cancer, sarcoma cancer, adenoma cancer, primitive neuroectodermal cancer (primitive neuroectodermal tumor (PNET)), and combinations thereof.

30. The antigen recognizing construct, or the T cell receptor, or the nucleic acid sequence, or the vector, or the host cell according to item 29, wherein the lung cancer is selected from the group consisting of squamous cell carcinoma of the lung, non-small cell lung cancer and small cell lung cancer.

31. The antigen recognizing construct, or the T cell receptor, or the nucleic acid sequence, or the vector, or the host cell according to item 29, wherein the breast cancer is selected from the group consisting of ductal breast cancer, tubular breast cancer, medullary breast cancer and combinations thereof.

32. The antigen recognizing construct, or the T cell receptor, or the nucleic acid sequence, or the vector, or the host cell according to item 29, wherein the gastric cancer is gastric adenocarcinoma cancer.

33. The antigen recognizing construct, or the T cell receptor, or the nucleic acid sequence, or the vector, or the host cell according to item 29, wherein the sarcoma cancer is selected from the group consisting of chondrosarcoma cancer, osteosarcoma cancer and combinations thereof.

34. The antigen recognizing construct, or the T cell receptor, or the nucleic acid sequence, or the vector, or the host cell according to item 29, wherein the adenoma cancer is selected from the group consisting of gastric adenocarcinoma, pancreatic adenocarcinoma and combinations thereof.

35. Use of the antigen recognizing construct or the T cell receptor according to any one of items 1 to 20, or the nucleic acid sequence according to item 21, or the vector according to item 22, or the host cell according to item 23, for the manufacture of a medicament for treating a disease.

36. The use of the antigen recognizing construct or the T cell receptor, or the nucleic acid sequence, or the vector, or the host cell according to item 35, wherein the disease is a malignant or benign tumor disease.

37. The use of the antigen recognizing construct or the T cell receptor, or the nucleic acid sequence, or the vector, or the host cell, according to item 35 or item 36, wherein the disease is a tumor that expresses mutated KRAS.

38. The use of the antigen recognizing construct or the T cell receptor, or the nucleic acid sequence, or the vector, or the host cell according to item 37, wherein the disease is an advanced-stage tumor that expresses mutated KRAS.

39. The use of the antigen recognizing construct or the T cell receptor, or the nucleic acid sequence, or the vector, or the host cell according to any one of items 35 to 38, wherein the disease is selected from the group consisting of Hodgkin's lymphoma, non-Hodgkin's lymphoma, acute myeloid leukemia, pancreatic cancer, colorectal cancer, endometrial cancer, biliary tract cancer, liver cancer, myeloma, prostate cancer, stomach cancer, kidney cancer, bone cancer, soft tissue cancer, head and neck cancer, glioblastoma multiforme, astrocytomas, melanoma, lung cancer, esophageal cancer, gastric cancer, breast cancer, ovarian cancer, mesothelioma cancer, bladder cancer, anal cancer, chondrosarcoma cancer, osteosarcoma cancer, sarcoma cancer, adenoma cancer, primitive neuroectodermal cancer (primitive neuroectodermal tumor (PNET)), and combinations thereof.

40. The use of the antigen recognizing construct or the T cell receptor, or the nucleic acid sequence, or the vector, or the host cell according to item 39, wherein the lung cancer is selected from the group consisting of squamous cell carcinoma of the lung, non-small cell lung cancer and small cell lung cancer.

41. The use of the antigen recognizing construct or the T cell receptor, or the nucleic acid sequence, or the vector, or the host cell according to item 39, wherein the breast cancer is selected from the group consisting of ductal breast cancer, tubular breast cancer, medullary breast cancer and combinations thereof.

42. The use of the antigen recognizing construct or the T cell receptor, or the nucleic acid sequence, or the vector, or the host cell according to item 39, wherein the gastric cancer is gastric adenocarcinoma cancer.

43. The use of the antigen recognizing construct or the T cell receptor, or the nucleic acid sequence, or the vector, or the host cell according to item 39, wherein the sarcoma cancer is selected from the group consisting of chondrosarcoma cancer, osteosarcoma cancer and combinations thereof.

44. The use of the antigen recognizing construct or the T cell receptor, or the nucleic acid sequence, or the vector, or the host cell according to item 39, wherein the adenoma cancer is selected from the group consisting of gastric adenocarcinoma, pancreatic adenocarcinoma and combinations thereof.

45. A method of treating a disease, comprising the step of administering a therapeutically effective amount of the antigen recognizing construct or the T cell receptor according to any one of items 1 to 20, or the nucleic acid sequence according to item 21, or the vector according to item 22, or the host cell according to item 23.

46. The method of treating a disease according to item 45, wherein the disease is a malignant or benign tumor disease.

47. The method of treating a disease according to item 45 or item 46, wherein the disease is a tumor that expresses mutated KRAS.

48. The method of treating a disease according to item 47, wherein the disease is an advanced-stage tumor that expresses mutated KRAS.

49. The method of treating a disease according to any one of items 45 to 48, wherein the disease is selected from the group consisting of Hodgkin's lymphoma, non-Hodgkin's lymphoma, acute myeloid leukemia, pancreatic cancer, colorectal cancer, endometrial cancer, biliary tract cancer, liver cancer, myeloma, prostate cancer, stomach cancer, kidney cancer, bone cancer, soft tissue cancer, head and neck cancer, glioblastoma multiforme, astrocytomas, melanoma, lung cancer, esophageal cancer, gastric cancer, breast cancer, ovarian cancer, mesothelioma cancer, bladder cancer, anal cancer, chondrosarcoma cancer, osteosarcoma cancer, sarcoma cancer, adenoma cancer, primitive neuroectodermal cancer (primitive neuroectodermal tumor (PNET)), and combinations thereof.

50. The method of treating a disease according to item 49, wherein the lung cancer is selected from the group consisting of squamous cell carcinoma of the lung, non-small cell lung cancer and small cell lung cancer.

51. The method of treating a disease according to item 49, wherein the breast cancer is selected from the group consisting of ductal breast cancer, tubular breast cancer, medullary breast cancer and combinations thereof.

52. The method of treating a disease according to item 49, wherein the gastric cancer is gastric adenocarcinoma cancer.

53. The method of treating a disease according to item 49, wherein the sarcoma cancer is selected from the group consisting of chondrosarcoma cancer, osteosarcoma cancer and combinations thereof.

54. The method of treating a disease according to item 49, wherein the adenoma cancer is selected from the group consisting of gastric adenocarcinoma, pancreatic adenocarcinoma and combinations thereof.

55. A pharmaceutical composition comprising the antigen recognizing construct or the T cell receptor according to any one of items 1 to 20, or the nucleic acid sequence according to item 21, or the vector according to item 22, or the host cell according to item 23.

56. A kit for use in medicine comprising the antigen recognizing construct or the T cell receptor according to any one of items 1 to 20, or the nucleic acid sequence according to item 21, or the vector according to item 22, or the host cell according to item 23.

57. The kit according to item 56, wherein the kit is a diagnostic kit for selecting a patient for treatment of a tumor, wherein cells of the tumor express mutated KRAS.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

As used herein, the term “about” means ±20% of the indicated range, value, or structure, unless otherwise indicated. It should be understood that the terms “a” and “an”, as used herein, refer to “one or more” of the enumerated components. The use of the alternative (e.g., “or”) should be understood to mean either one, both, or any combination thereof of the alternatives. As used herein, the terms “include”, “have”, and “comprise” are used synonymously, which terms and variants thereof are intended to be construed as non-limiting.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein and herein disclosed may be resorted to by those skilled in the art, and that such modifications and variations are considered to be within the scope of this invention. The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group. Further embodiments of the invention will become apparent from the following claims.

What is claimed is:

1. An antigen recognizing construct that binds the peptide VVGAVGVGK (SEQ ID NO: 1) with determinable affinity,
wherein the antigen recognizing construct comprises a complementary determining region 3 (CDR3) of the α-chain having at least 90% sequence identity to an amino acid sequence selected from the group consisting of SEQ ID NOs: 10, 16, 4, 22, and 28, and/or
wherein the antigen recognizing construct comprises a complementary determining region 3 (CDR3) of the β-chain having at least 90% sequence identity to an amino acid sequence selected from the group consisting of SEQ ID NOs: 13, 19, 7, 25, and 31.

2. The antigen recognizing construct of claim 1, wherein the antigen recognizing construct is an antibody, or fragment thereof, or a T cell receptor (TCR), or fragment thereof.

3. The antigen recognizing construct of claim 1 or claim 2, wherein the antigen recognizing construct specifically binds the peptide VVGAVGVGK (SEQ ID NO: 1) with an EC₅₀ of less than about 1×10⁻⁶ M.

4. The antigen recognizing construct of claim 3, wherein the EC₅₀ value is determined by the procedure as carried out in Example 2.

5. The antigen recognizing construct of any one of the preceding claims, wherein the antigen recognizing construct binds the peptide being presented by a MHC I molecule.

6. The antigen recognizing construct of claim 5, wherein the MHC I molecule is an HLA-A molecule.

7. The antigen recognizing construct of claim 6, wherein the HLA-A molecule is HLA-A*11:01.

8. The antigen recognizing construct of any one of the preceding claims, wherein the antigen recognizing construct binds the peptide VVGAVGVGK (SEQ ID NO: 1) with an antigenic specificity of at least 80%.

9. The antigen recognizing construct of any one of the preceding claims, wherein the antigen recognizing construct is a T cell receptor (TCR).

10. The T cell receptor of claim 9, wherein the T cell receptor comprises a complementary determining region 1 (CDR1) of the α-chain having at least 90% sequence identity to an amino acid sequence selected from the group consisting of SEQ ID NOs: 8, 14, 2, 20, and 26, and/or wherein the T cell receptor comprises a complementary determining region 1 (CDR1) of the β-chain having at least 90% sequence identity to an amino acid sequence selected from the group consisting of SEQ ID NOs: 11, 17, 5, 23, and 29.

11. The T cell receptor of claim 9 or 10, wherein the T cell receptor comprises a complementary determining region 2 (CDR2) of the α-chain having at least 90% sequence identity to an amino acid sequence selected from the group consisting of SEQ ID NOs: 9, 15, 3, 21, and 27, and/or wherein the T cell receptor comprises a complementary determining region 2 (CDR2) of the β-chain having at least 90% sequence identity to an amino acid sequence selected from the group consisting of SEQ ID NOs: 12, 18, 6, 24, and 30.

12. The T cell receptor of any one of claims 9 to 11, wherein the T cell receptor comprises a complementary determining region 3 (CDR3) of the α-chain having at least 95% sequence identity to an amino acid sequence selected from the group consisting of SEQ ID NOs: 10, 16, 4, 22, and 28, and/or wherein the T cell receptor comprises a complementary determining region 3 (CDR3) of the β-chain having at least 95% sequence identity to an amino acid sequence selected from the group consisting of SEQ ID NOs: 13, 19, 7, 25, and 31.

13. The T cell receptor of any one of claims 9 to 12, wherein the T cell receptor comprises a complementary determining region 1 (CDR1) of the α-chain having at least 95% sequence identity to an amino acid sequence selected from the group consisting of SEQ ID NOs: 8, 14, 2, 20, and 26, and/or wherein the T cell receptor comprises a complementary determining region 1 (CDR1) of the β-chain having at least 95% sequence identity to an amino acid sequence selected from the group consisting of SEQ ID NOs: 11, 17, 5, 23, and 29.

14. The T cell receptor of any one of claims 9 to 13, wherein the T cell receptor comprises a complementary determining region 2 (CDR2) of the α-chain having at least 95% sequence identity to an amino acid sequence selected from the group consisting of SEQ ID NOs: 9, 15, 3, 21, and 27, and/or wherein the T cell receptor comprises a complementary determining region 2 (CDR2) of the β-chain having at least 95% sequence identity to an amino acid sequence selected from the group consisting of SEQ ID NOs: 12, 18, 6, 24, and 30.

15. The T cell receptor of any one of claims 9 to 14, comprising:
(a) an α-chain CDR1 comprising or consisting of the amino acid sequence of SEQ ID NO: 8,
an α-chain CDR2 comprising or consisting of the amino acid sequence of SEQ ID NO: 9, and
an α-chain CDR3 comprising or consisting of the amino acid sequence of SEQ ID NO:
10; or
(b) an α-chain CDR1 comprising or consisting of the amino acid sequence of SEQ ID NO: 14,
an α-chain CDR2 comprising or consisting of the amino acid sequence of SEQ ID NO:
15, and
an α-chain CDR3 comprising or consisting of the amino acid sequence of SEQ ID NO:
16; or
(c) an α-chain CDR1 comprising or consisting of the amino acid sequence of SEQ ID NO: 2,
an α-chain CDR2 comprising or consisting of the amino acid sequence of SEQ ID NO: 3, and
an α-chain CDR3 comprising or consisting of the amino acid sequence of SEQ ID NO: 4; or
(d) an α-chain CDR1 comprising or consisting of the amino acid sequence of SEQ ID NO: 20,
an α-chain CDR2 comprising or consisting of the amino acid sequence of SEQ ID NO: 21, and
an α-chain CDR3 comprising or consisting of the amino acid sequence of SEQ ID NO: 22; or
(e) an α-chain CDR1 comprising or consisting of the amino acid sequence of SEQ ID NO: 26,
an α-chain CDR2 comprising or consisting of the amino acid sequence of SEQ ID NO: 27, and
an α-chain CDR3 comprising or consisting of the amino acid sequence of SEQ ID NO: 28.

16. The T cell receptor of any one of claims 9 to 15, comprising:
(a) a β-chain CDR1 comprising or consisting of the amino acid sequence of SEQ ID NO: 11,
a β-chain CDR2 comprising or consisting of the amino acid sequence of SEQ ID NO: 12, and
a β-chain CDR3 comprising or consisting of the amino acid sequence of SEQ ID NO: 13; or
(b) a β-chain CDR1 comprising or consisting of the amino acid sequence of SEQ ID NO: 17,
a β-chain CDR2 comprising or consisting of the amino acid sequence of SEQ ID NO: 18, and
a β-chain CDR3 comprising or consisting of the amino acid sequence of SEQ ID NO: 19; or
(c) a β-chain CDR1 comprising or consisting of the amino acid sequence of SEQ ID NO: 5,
a β-chain CDR2 comprising or consisting of the amino acid sequence of SEQ ID NO: 6, and
a β-chain CDR3 comprising or consisting of the amino acid sequence of SEQ ID NO: 7; or
(d) a β-chain CDR1 comprising or consisting of the amino acid sequence of SEQ ID NO: 23,
a β-chain CDR2 comprising or consisting of the amino acid sequence of SEQ ID NO: 24, and
a β-chain CDR3 comprising or consisting of the amino acid sequence of SEQ ID NO: 25; or
(e) a β-chain CDR1 comprising or consisting of the amino acid sequence of SEQ ID NO: 29,
a β-chain CDR2 comprising or consisting of the amino acid sequence of SEQ ID NO: 30, and
a β-chain CDR3 comprising or consisting of the amino acid sequence of SEQ ID NO: 31.

17. The T cell receptor of any one of claims 9 to 16, comprising:
(a) an α-chain CDR1 comprising or consisting of the amino acid sequence of SEQ ID NO: 8,
an α-chain CDR2 comprising or consisting of the amino acid sequence of SEQ ID NO: 9, an α-chain CDR3 comprising or consisting of the amino acid sequence of SEQ ID NO: 10;
a β-chain CDR1 comprising or consisting of the amino acid sequence of SEQ ID NO: 11,
a β-chain CDR2 comprising or consisting of the amino acid sequence of SEQ ID NO: 12, and
a β-chain CDR3 comprising or consisting of the amino acid sequence of SEQ ID NO: 13; or
(b) an α-chain CDR1 comprising or consisting of the amino acid sequence of SEQ ID NO: 14,
an α-chain CDR2 comprising or consisting of the amino acid sequence of SEQ ID NO: 15,
an α-chain CDR3 comprising or consisting of the amino acid sequence of SEQ ID NO: 16;
a β-chain CDR1 comprising or consisting of the amino acid sequence of SEQ ID NO: 17,
a β-chain CDR2 comprising or consisting of the amino acid sequence of SEQ ID NO: 18, and
a β-chain CDR3 comprising or consisting of the amino acid sequence of SEQ ID NO: 19; or
(c) an α-chain CDR1 comprising or consisting of the amino acid sequence of SEQ ID NO: 2,
an α-chain CDR2 comprising or consisting of the amino acid sequence of SEQ ID NO: 3,
an α-chain CDR3 comprising or consisting of the amino acid sequence of SEQ ID NO: 4;
a β-chain CDR1 comprising or consisting of the amino acid sequence of SEQ ID NO: 5,
a β-chain CDR2 comprising or consisting of the amino acid sequence of SEQ ID NO: 6, and
a β-chain CDR3 comprising or consisting of the amino acid sequence of SEQ ID NO: 7; or
(d) an α-chain CDR1 comprising or consisting of the amino acid sequence of SEQ ID NO: 20,
an α-chain CDR2 comprising or consisting of the amino acid sequence of SEQ ID NO: 21,
an α-chain CDR3 comprising or consisting of the amino acid sequence of SEQ ID NO: 22;
a β-chain CDR1 comprising or consisting of the amino acid sequence of SEQ ID NO: 23,
a β-chain CDR2 comprising or consisting of the amino acid sequence of SEQ ID NO: 24, and
a β-chain CDR3 comprising or consisting of the amino acid sequence of SEQ ID NO: 25; or
(e) an α-chain CDR1 comprising or consisting of the amino acid sequence of SEQ ID NO: 26,
an α-chain CDR2 comprising or consisting of the amino acid sequence of SEQ ID NO: 27,
an α-chain CDR3 comprising or consisting of the amino acid sequence of SEQ ID NO: 28;
a β-chain CDR1 comprising or consisting of the amino acid sequence of SEQ ID NO: 29, a β-chain CDR2 comprising or consisting of the amino acid sequence of SEQ ID NO: 30, and
a β-chain CDR3 comprising or consisting of the amino acid sequence of SEQ ID NO: 31.

18. The T cell receptor of any one of claims 10 to 17, comprising:
(a) an α chain comprising CDR sequences comprising amino acid sequences of SEQ ID NOs: 8, 9, and 10, and a β chain comprising CDR sequences comprising amino acid sequences of SEQ ID NOs: 11, 12, and 13; or
(b) an α chain comprising CDR sequences comprising amino acid sequences of SEQ ID NOs: 14, 15, and 16, and a β chain comprising CDR sequences comprising amino acid sequences of SEQ ID NOs: 17, 18, and 19; or
(c) an α chain comprising CDR sequences comprising amino acid sequences of SEQ ID NOs: 2, 3, and 4, and a β chain comprising CDR sequences comprising amino acid sequences of SEQ ID NOs: 5, 6, and 7; or
(d) an α chain comprising CDR sequences comprising amino acid sequences of SEQ ID NOs: 20, 21, and 22, and a β chain comprising CDR sequences comprising amino acid sequences of SEQ ID NOs: 23, 24, and 25; or
(e) an α chain comprising CDR sequences comprising amino acid sequences of SEQ ID NO: 26, 27, and 28, and a β chain comprising CDR sequences comprising amino acid sequences of SEQ ID NOs: 29, 30, and 31.

19. The T cell receptor of any one of claims 9 to 18, comprising:
(a) an amino acid sequence having at least 80%, at least 85%, at least 90%, at least 95% or at least 99% sequence identity to the variable domain of SEQ ID NO: 32, and/or an amino acid sequence having at least 80%, at least 85%, at least 90%, at least 95% or at least 99% sequence identity to the variable domain of SEQ ID NO: 33; or
(b) an amino acid sequence having at least 80%, at least 85%, at least 90%, at least 95% or at least 99% sequence identity to the variable domain of SEQ ID NO: 34, and/or an amino acid sequence having at least 80%, at least 85%, at least 90%, at least 95% or at least 99% sequence identity to the variable domain of SEQ ID NO: 35; or
(c) an amino acid sequence having at least 80%, at least 85%, at least 90%, at least 95% or at least 99% sequence identity to the variable domain of SEQ ID NO: 36, and/or an amino acid sequence having at least 80%, at least 85%, at least 90%, at least 95% or at least 99% sequence identity to the variable domain of SEQ ID NO: 37; or
(d) an amino acid sequence having at least 80%, at least 85%, at least 90%, at least 95% or at least 99% sequence identity to the variable domain of SEQ ID NO: 38, and/or an amino acid sequence having at least 80%, at least 85%, at least 90%, at least 95% or at least 99% sequence identity to the variable domain of SEQ ID NO: 39; or
(e) an amino acid sequence having at least 80%, at least 85%, at least 90%, at least 95% or at least 99% sequence identity to the variable domain of SEQ ID NO: 40, and/or an amino acid sequence having at least 80%, at least 85%, at least 90%, at least 95% or at least 99% sequence identity to the variable domain of SEQ ID NO: 41.

20. The T cell receptor of any one of claims 9 to 19, comprising:
(a) an α chain comprising a sequence having at least 80%, at least 85%, at least 90%, at least 95% or at least 99% sequence identity to the amino acid sequence of SEQ ID NO: 32, and/or a β chain comprising a sequence having at least 80%, at least 85%, at least 90%, at least 95% or at least 99% sequence identity to the amino acid sequence of SEQ ID NO: 33; or
(b) an α chain comprising a sequence having at least 80%, at least 85%, at least 90%, at least 95% or at least 99% sequence identity to the amino acid sequence of SEQ ID NO: 34, and/or a β chain comprising a sequence having at least 80%, at least 85%, at least 90%, at least 95% or at least 99% sequence identity to the amino acid sequence of SEQ ID NO: 35; or
(c) an α chain comprising a sequence having at least 80%, at least 85%, at least 90%, at least 95% or at least 99% sequence identity to the amino acid sequence of SEQ ID NO: 36, and/or a β chain comprising a sequence having at least 80%, at least 85%, at least 90%, at least 95% or at least 99% sequence identity to the amino acid sequence of SEQ ID NO: 37; or
(d) an α chain comprising a sequence having at least 80%, at least 85%, at least 90%, at least 95% or at least 99% sequence identity to the amino acid sequence of SEQ ID NO: 38, and/or a β chain comprising a sequence having at least 80%, at least 85%, at least 90%, at least 95% or at least 99% sequence identity to the amino acid sequence of SEQ ID NO: 39; or
(e) an α chain comprising a sequence having at least 80%, at least 85%, at least 90%, at least 95% or at least 99% sequence identity to the amino acid sequence of SEQ ID NO: 40, and/or a β chain comprising a sequence having at least 80%, at least 85%, at least 90%, at least 95% or at least 99% sequence identity to the amino acid sequence of SEQ ID NO: 41.

21. A T cell receptor (TCR) having antigenic specificity for mutated KRAS, wherein the TCR comprises:
(a) an α-chain CDR1 comprising or consisting of the amino acid sequence of SEQ ID NO: 8,
an α-chain CDR2 comprising or consisting of the amino acid sequence of SEQ ID NO: 9, and
an α-chain CDR3 comprising or consisting of the amino acid sequence of SEQ ID NO: 10; or
(b) an α-chain CDR1 comprising or consisting of the amino acid sequence of SEQ ID NO: 14,
an α-chain CDR2 comprising or consisting of the amino acid sequence of SEQ ID NO: 15, and
an α-chain CDR3 comprising or consisting of the amino acid sequence of SEQ ID NO: 16; or
(c) an α-chain CDR1 comprising or consisting of the amino acid sequence of SEQ ID NO: 2,
an α-chain CDR2 comprising or consisting of the amino acid sequence of SEQ ID NO: 3, and
an α-chain CDR3 comprising or consisting of the amino acid sequence of SEQ ID NO: 4; or
(d) an α-chain CDR1 comprising or consisting of the amino acid sequence of SEQ ID NO: 20,
an α-chain CDR2 comprising or consisting of the amino acid sequence of SEQ ID NO: 21, and
an α-chain CDR3 comprising or consisting of the amino acid sequence of SEQ ID NO: 22; or
(e) an α-chain CDR1 comprising or consisting of the amino acid sequence of SEQ ID NO: 26,
an α-chain CDR2 comprising or consisting of the amino acid sequence of SEQ ID NO: 27, and
an α-chain CDR3 comprising or consisting of the amino acid sequence of SEQ ID NO: 28.

22. A T cell receptor (TCR) having antigenic specificity for mutated KRAS, wherein the TCR comprises:
(a) a β-chain CDR1 comprising or consisting of the amino acid sequence of SEQ ID NO: 11,
a β-chain CDR2 comprising or consisting of the amino acid sequence of SEQ ID NO: 12, and
a β-chain CDR3 comprising or consisting of the amino acid sequence of SEQ ID NO: 13; or
(b) a β-chain CDR1 comprising or consisting of the amino acid sequence of SEQ ID NO: 17,
a β-chain CDR2 comprising or consisting of the amino acid sequence of SEQ ID NO: 18, and
a β-chain CDR3 comprising or consisting of the amino acid sequence of SEQ ID NO: 19; or
(c) a β-chain CDR1 comprising or consisting of the amino acid sequence of SEQ ID NO: 5,
a β-chain CDR2 comprising or consisting of the amino acid sequence of SEQ ID NO: 6, and
a β-chain CDR3 comprising or consisting of the amino acid sequence of SEQ ID NO: 7; or
(d) a β-chain CDR1 comprising or consisting of the amino acid sequence of SEQ ID NO: 23,
a β-chain CDR2 comprising or consisting of the amino acid sequence of SEQ ID NO: 24, and
a β-chain CDR3 comprising or consisting of the amino acid sequence of SEQ ID NO: 25; or
(e) a β-chain CDR1 comprising or consisting of the amino acid sequence of SEQ ID NO: 29,
a β-chain CDR2 comprising or consisting of the amino acid sequence of SEQ ID NO: 30, and
a β-chain CDR3 comprising or consisting of the amino acid sequence of SEQ ID NO: 31.

23. A T cell receptor (TCR) having antigenic specificity for mutated KRAS, wherein the TCR comprises:
(a) an α-chain CDR1 comprising or consisting of the amino acid sequence of SEQ ID NO: 8,
an α-chain CDR2 comprising or consisting of the amino acid sequence of SEQ ID NO: 9,
an α-chain CDR3 comprising or consisting of the amino acid sequence of SEQ ID NO: 10;
a β-chain CDR1 comprising or consisting of the amino acid sequence of SEQ ID NO: 11,
a β-chain CDR2 comprising or consisting of the amino acid sequence of SEQ ID NO: 12, and
a β-chain CDR3 comprising or consisting of the amino acid sequence of SEQ ID NO: 13; or
(b) an α-chain CDR1 comprising or consisting of the amino acid sequence of SEQ ID NO: 14,
an α-chain CDR2 comprising or consisting of the amino acid sequence of SEQ ID NO: 15,
an α-chain CDR3 comprising or consisting of the amino acid sequence of SEQ ID NO: 16;
a β-chain CDR1 comprising or consisting of the amino acid sequence of SEQ ID NO: 17,
a β-chain CDR2 comprising or consisting of the amino acid sequence of SEQ ID NO: 18, and
a β-chain CDR3 comprising or consisting of the amino acid sequence of SEQ ID NO: 19; or
(c) an α-chain CDR1 comprising or consisting of the amino acid sequence of SEQ ID NO: 2,
an α-chain CDR2 comprising or consisting of the amino acid sequence of SEQ ID NO: 3,
an α-chain CDR3 comprising or consisting of the amino acid sequence of SEQ ID NO: 4,
a β-chain CDR1 comprising or consisting of the amino acid sequence of SEQ ID NO: 5,
a β-chain CDR2 comprising or consisting of the amino acid sequence of SEQ ID NO: 6, and
a β-chain CDR3 comprising or consisting of the amino acid sequence of SEQ ID NO: 7; or
(d) an α-chain CDR1 comprising or consisting of the amino acid sequence of SEQ ID NO: 20,
an α-chain CDR2 comprising or consisting of the amino acid sequence of SEQ ID NO: 21,
an α-chain CDR3 comprising or consisting of the amino acid sequence of SEQ ID NO: 22;
a β-chain CDR1 comprising or consisting of the amino acid sequence of SEQ ID NO: 23,
a β-chain CDR2 comprising or consisting of the amino acid sequence of SEQ ID NO: 24, and
a β-chain CDR3 comprising or consisting of the amino acid sequence of SEQ ID NO: 25; or
(e) an α-chain CDR1 comprising or consisting of the amino acid sequence of SEQ ID NO: 26,
an α-chain CDR2 comprising or consisting of the amino acid sequence of SEQ ID NO: 27,
an α-chain CDR3 comprising or consisting of the amino acid sequence of SEQ ID NO: 28;
a β-chain CDR1 comprising or consisting of the amino acid sequence of SEQ ID NO: 29,
a β-chain CDR2 comprising or consisting of the amino acid sequence of SEQ ID NO: 30, and
a β-chain CDR3 comprising or consisting of the amino acid sequence of SEQ ID NO: 31.

24. A T cell receptor (TCR) having antigenic specificity for mutated KRAS, wherein the TCR comprises:
(a) an α chain comprising CDR sequences comprising amino acid sequences of SEQ ID NOs: 8, 9, and 10, and a β chain comprising CDR sequences comprising amino acid sequences of SEQ ID NOs: 11, 12, and 13; or
(b) an α chain comprising CDR sequences comprising amino acid sequences of SEQ ID NOs: 14, 15, and 16, and a β chain comprising CDR sequences comprising amino acid sequences of SEQ ID NOs: 17, 18, and 19; or
(c) an α chain comprising CDR sequences comprising amino acid sequences of SEQ ID NOs: 2, 3, and 4, and a β chain comprising CDR sequences comprising amino acid sequences of SEQ ID NOs: 5, 6, and 7; or
(d) an α chain comprising CDR sequences comprising amino acid sequences of SEQ ID NOs: 20, 21, and 22, and a β chain comprising CDR sequences comprising amino acid sequences of SEQ ID NOs: 23, 24, and 25; or
(e) an α chain comprising CDR sequences comprising amino acid sequences of SEQ ID NO: 26, 27, and 28, and a β chain comprising CDR sequences comprising amino acid sequences of SEQ ID NOs: 29, 30, and 31.

25. A T cell receptor (TCR) having antigenic specificity for mutated KRAS, wherein the TCR comprises:
(a) an amino acid sequence having at least 80%, at least 85%, at least 90%, at least 95% or at least 99% sequence identity to the variable domain of SEQ ID NO: 32, and/or an amino acid sequence having at least 80%, at least 85%, at least 90%, at least 95% or at least 99% sequence identity to the variable domain of SEQ ID NO: 33; or
(b) an amino acid sequence having at least 80%, at least 85%, at least 90%, at least 95% or at least 99% sequence identity to the variable domain of SEQ ID NO: 34, and/or an amino acid sequence having at least 80%, at least 85%, at least 90%, at least 95% or at least 99% sequence identity to the variable domain of SEQ ID NO: 35; or
(c) an amino acid sequence having at least 80%, at least 85%, at least 90%, at least 95% or at least 99% sequence identity to the variable domain of SEQ ID NO: 36, and/or an amino acid sequence having at least 80%, at least 85%, at least 90%, at least 95% or at least 99% sequence identity to the variable domain of SEQ ID NO: 37; or
(d) an amino acid sequence having at least 80%, at least 85%, at least 90%, at least 95% or at least 99% sequence identity to the variable domain of SEQ ID NO: 38, and/or an amino acid sequence having at least 80%, at least 85%, at least 90%, at least 95% or at least 99% sequence identity to the variable domain of SEQ ID NO: 39; or
(e) an amino acid sequence having at least 80%, at least 85%, at least 90%, at least 95% or at least 99% sequence identity to the variable domain of SEQ ID NO: 40, and/or an amino acid sequence having at least 80%, at least 85%, at least 90%, at least 95% or at least 99% sequence identity to the variable domain of SEQ ID NO: 41.

26. A T cell receptor (TCR) having antigenic specificity for mutated KRAS, wherein the TCR comprises:
(a) an α chain comprising a sequence having at least 80%, at least 85%, at least 90%, at least 95% or at least 99% sequence identity to the amino acid sequence of SEQ ID NO: 32, and/or a β chain comprising a sequence having at least 80%, at least 85%, at least 90%, at least 95% or at least 99% sequence identity to the amino acid sequence of SEQ ID NO: 33; or
(b) an α chain comprising a sequence having at least 80%, at least 85%, at least 90%, at least 95% or at least 99% sequence identity to the amino acid sequence of SEQ ID NO: 34, and/or a β chain comprising a sequence having at least 80%, at least 85%, at least 90%, at least 95% or at least 99% sequence identity to the amino acid sequence of SEQ ID NO: 35; or
(c) an α chain comprising a sequence having at least 80%, at least 85%, at least 90%, at least 95% or at least 99% sequence identity to the amino acid sequence of SEQ ID NO: 36, and/or a β chain comprising a sequence having at least 80%, at least 85%, at least 90%, at least 95% or at least 99% sequence identity to the amino acid sequence of SEQ ID NO: 37; or
(d) an α chain comprising a sequence having at least 80%, at least 85%, at least 90%, at least 95% or at least 99% sequence identity to the amino acid sequence of SEQ ID NO: 38, and/or a β chain comprising a sequence having at least 80%, at least 85%, at least 90%, at least 95% or at least 99% sequence identity to the amino acid sequence of SEQ ID NO: 39; or
(e) an α chain comprising a sequence having at least 80%, at least 85%, at least 90%, at least 95% or at least 99% sequence identity to the amino acid sequence of SEQ ID NO: 40, and/or a β chain comprising a sequence having at least 80%, at least 85%, at least 90%, at least 95% or at least 99% sequence identity to the amino acid sequence of SEQ ID NO: 41.

27. A nucleic acid sequence encoding an antigen recognizing construct or T cell receptor as defined in any one of claims 1 to 26.

28. A vector comprising a nucleic acid sequence as defined in claim 27.

29. A host cell comprising the antigen recognizing construct or T cell receptor according to any one of claims 1 to 26, or the nucleic acid sequence according to claim 27, or the vector according to claim 28.

30. The antigen recognizing construct or the T cell receptor according to any one of claims 1 to 26, or the nucleic acid sequence according to claim 27, or the vector according to claim 28, or the host cell according to claim 29, for use in medicine.

31. The antigen recognizing construct or the T cell receptor according to any one of claims 1 to 26, or the nucleic acid sequence according to claim 27, or the vector according to claim 28, or the host cell according to claim 29, for use in the prevention and/or treatment of a disease.

32. The antigen recognizing construct, or the T cell receptor, or the nucleic acid sequence, or the vector, or the host cell according to claim 31, wherein the disease is a malignant or benign tumor disease.

33. The antigen recognizing construct, or the T cell receptor, or the nucleic acid sequence, or the vector, or the host cell according to claim 31 or claim 32, wherein the disease is a tumor that expresses mutated KRAS.

34. The antigen recognizing construct, or the T cell receptor, or the nucleic acid sequence, or the vector, or the host cell according to claim 33, wherein the disease is an advanced-stage tumor that expresses mutated KRAS.

35. The antigen recognizing construct, or the T cell receptor, or the nucleic acid sequence, or the vector, or the host cell according to any one of claims 31 to 34, wherein the disease is selected from the group consisting of Hodgkin's lymphoma, non-Hodgkin's lymphoma, acute myeloid leukemia, pancreatic cancer, colorectal cancer, endometrial cancer, biliary tract cancer, liver cancer, myeloma, prostate cancer, stomach cancer, kidney cancer, bone cancer, soft tissue cancer, head and neck cancer, glioblastoma multiforme, astrocytomas, melanoma, lung cancer, esophageal cancer, gastric cancer, breast cancer, ovarian cancer, mesothelioma cancer, bladder cancer, anal cancer, chondrosarcoma cancer, osteosarcoma cancer, sarcoma cancer, adenoma cancer, primitive neuroectodermal cancer (primitive neuroectodermal tumor (PNET)), and combinations thereof.

36. The antigen recognizing construct, or the T cell receptor, or the nucleic acid sequence, or the vector, or the host cell according to claim 35, wherein the lung cancer is selected from the group consisting of squamous cell carcinoma of the lung, non-small cell lung cancer and small cell lung cancer.

37. The antigen recognizing construct, or the T cell receptor, or the nucleic acid sequence, or the vector, or the host cell according to claim 35, wherein the breast cancer is selected from the group consisting of ductal breast cancer, tubular breast cancer, medullary breast cancer and combinations thereof.

38. The antigen recognizing construct, or the T cell receptor, or the nucleic acid sequence, or the vector, or the host cell according to claim 35, wherein the gastric cancer is gastric adenocarcinoma cancer.

39. The antigen recognizing construct, or the T cell receptor, or the nucleic acid sequence, or the vector, or the host cell according to claim 35, wherein the sarcoma cancer is selected from the group consisting of chondrosarcoma cancer, osteosarcoma cancer and combinations thereof.

40. The antigen recognizing construct, or the T cell receptor, or the nucleic acid sequence, or the vector, or the host cell according to claim 35, wherein the adenoma cancer is selected from the group consisting of gastric adenocarcinoma, pancreatic adenocarcinoma and combinations thereof.

41. Use of the antigen recognizing construct or the T cell receptor according to any one of claims 1 to 26, or the nucleic acid sequence according to claim 27, or the vector according to claim 28, or the host cell according to claim 29, for the manufacture of a medicament for treating a disease.

42. The use of the antigen recognizing construct or the T cell receptor, or the nucleic acid sequence, or the vector, or the host cell according to claim 41, wherein the disease is a malignant or benign tumor disease.

43. The use of the antigen recognizing construct or the T cell receptor, or the nucleic acid sequence, or the vector, or the host cell, according to claim 41 or claim 42, wherein the disease is a tumor that expresses mutated KRAS.

44. The use of the antigen recognizing construct or the T cell receptor, or the nucleic acid sequence, or the vector, or the host cell according to claim 43, wherein the disease is an advanced-stage tumor that expresses mutated KRAS.

45. The use of the antigen recognizing construct or the T cell receptor, or the nucleic acid sequence, or the vector, or the host cell according to any one of claims 41 to 44, wherein the disease is selected from the group consisting of Hodgkin's lymphoma, non-Hodgkin's lymphoma, acute myeloid leukemia, pancreatic cancer, colorectal cancer, endometrial cancer, biliary tract cancer, liver cancer, myeloma, prostate cancer, stomach cancer, kidney cancer, bone cancer, soft tissue cancer, head and neck cancer, glioblastoma multiforme, astrocytomas, melanoma, lung cancer, esophageal cancer, gastric cancer, breast cancer, ovarian cancer, mesothelioma cancer, bladder cancer, anal cancer, chondrosarcoma cancer, osteosarcoma cancer, sarcoma cancer, adenoma cancer, primitive neuroectodermal cancer (primitive neuroectodermal tumor (PNET)), and combinations thereof.

46. The use of the antigen recognizing construct or the T cell receptor, or the nucleic acid sequence, or the vector, or the host cell according to claim 45, wherein the lung cancer is selected from the group consisting of squamous cell carcinoma of the lung, non-small cell lung cancer and small cell lung cancer.

47. The use of the antigen recognizing construct or the T cell receptor, or the nucleic acid sequence, or the vector, or the host cell according to claim 45, wherein the breast cancer is selected from the group consisting of ductal breast cancer, tubular breast cancer, medullary breast cancer and combinations thereof.

48. The use of the antigen recognizing construct or the T cell receptor, or the nucleic acid sequence, or the vector, or the host cell according to claim 45, wherein the gastric cancer is gastric adenocarcinoma cancer.

49. The use of the antigen recognizing construct or the T cell receptor, or the nucleic acid sequence, or the vector, or the host cell according to claim 45, wherein the sarcoma cancer is selected from the group consisting of chondrosarcoma cancer, osteosarcoma cancer and combinations thereof.

50. The use of the antigen recognizing construct or the T cell receptor, or the nucleic acid sequence, or the vector, or the host cell according to claim 45, wherein the adenoma cancer is selected from the group consisting of gastric adenocarcinoma, pancreatic adenocarcinoma and combinations thereof.

51. A method of treating a disease, comprising the step of administering a therapeutically effective amount of the antigen recognizing construct or the T cell receptor according to any one of claims 1 to 26, or the nucleic acid sequence according to claim 27, or the vector according to claim 28, or the host cell according to claim 29.

52. The method of treating a disease according to claim 51, wherein the disease is a malignant or benign tumor disease.

53. The method of treating a disease according to claim 51 or claim 52, wherein the disease is a tumor that expresses mutated KRAS.

54. The method of treating a disease according to claim 53, wherein the disease is an advanced-stage tumor that expresses mutated KRAS.

55. The method of treating a disease according to any one of claims 51 to 54, wherein the disease is selected from the group consisting of Hodgkin's lymphoma, non-Hodgkin's lymphoma, acute myeloid leukemia, pancreatic cancer, colorectal cancer, endometrial cancer, biliary tract cancer, liver cancer, myeloma, prostate cancer, stomach cancer, kidney cancer, bone cancer, soft tissue cancer, head and neck cancer, glioblastoma multiforme, astrocytomas, melanoma, lung cancer, esophageal cancer, gastric cancer, breast cancer, ovarian cancer, mesothelioma cancer, bladder cancer, anal cancer, chondrosarcoma cancer, osteosarcoma cancer, sarcoma cancer, adenoma cancer, primitive neuroectodermal cancer (primitive neuroectodermal tumor (PNET)), and combinations thereof.

56. The method of treating a disease according to claim 55, wherein the lung cancer is selected from the group consisting of squamous cell carcinoma of the lung, non-small cell lung cancer and small cell lung cancer.

57. The method of treating a disease according to claim 55, wherein the breast cancer is selected from the group consisting of ductal breast cancer, tubular breast cancer, medullary breast cancer and combinations thereof.

58. The method of treating a disease according to claim 55, wherein the gastric cancer is gastric adenocarcinoma cancer.

59. The method of treating a disease according to claim 55, wherein the sarcoma cancer is selected from the group consisting of chondrosarcoma cancer, osteosarcoma cancer and combinations thereof.

60. The method of treating a disease according to claim 55, wherein the adenoma cancer is selected from the group consisting of gastric adenocarcinoma, pancreatic adenocarcinoma and combinations thereof.

61. A pharmaceutical composition comprising the antigen recognizing construct or the T cell receptor according to any one of claims 1 to 26, or the nucleic acid sequence according to claim 27, or the vector according to claim 28, or the host cell according to claim 29.

62. A kit for use in medicine comprising the antigen recognizing construct or the T cell receptor according to any one of claims 1 to 26, or the nucleic acid sequence according to claim 27, or the vector according to claim 28, or the host cell according to claim 29.

63. The kit according to claim 62, wherein the kit is a diagnostic kit for selecting a patient for treatment of a tumor, wherein cells of the tumor express mutated KRAS.
